(19) United States
(12) Patent Application Publication
(10) Pub. No.: US 2026/0237479 A1
(43) Pub. Date: Aug. 13, 2026

(54) SYSTEMS AND METHODS FOR GENERATING ELECTRONIC MEDICAL REPORTS USING POSITRON EMISSION TOMOGRAPHY OR SINGLE-PHOTON EMISSION COMPUTED TOMOGRAPHY WITH COMPUTED TOMOGRAPHY IMAGES

(71) Applicant: NUCS AI INC., Dover, DE (US)

(72) Inventor: Andrei GAFITA, Baltimore, MD (US)

(73) Assignee: NUCS AI INC., Dover, DE (US)

(21) Appl. No.: 19/639,495

(22) Filed: Apr. 6, 2026

Related U.S. Application Data
(63) Continuation-in-part of application No. PCT/IB2023/060020, filed on Oct. 5, 2023.

(51) Int. Cl. G16H 15/00; G06T 7/00; G06V 10/26; G06V 10/764; G06V 10/82; G16H 30/20

(52) U.S. Cl. CPC G16H 15/00; G06T 7/0012; G06V 10/764; G16H 30/20; G06T 2207/10081; G06T 2207/10104; G06T 2207/10108; G06T 2207/20084; G06T 2207/30096; G06V 10/26; G06V 10/82; G06V 2201/03

(57) ABSTRACT

Methods and systems are described herein for generating electronic medical reports using positron emission tomography or single-photon emission computed tomography with computed tomography images. The system may import, based on a patient identifier, clinical patient data from a record database and digital imaging data comprising image data and acquisition metadata. The system may process the image data by executing a first predictive model to generate an output image representing segmentations of cancer lesions from non-cancerous tissue. The system may extract quantitative tumor characteristics representing a cancer stage in accordance with a predefined staging classification. The system may generate, by executing a second predictive model, a prognostic or therapy-response prediction based on the quantitative tumor characteristics and clinical patient data. The system may generate an electronic medical report by populating a predefined template with the clinical patient data, acquisition metadata, cancer stage, and quantitative tumor characteristics.

### CROSS-REFERENCE TO RELATED APPLICATIONS

The present disclosure is a continuation-in-part of PCT/IB2023/060020 titled “A PROCESS FOR AUTOMATIC GENERATION OF STRUCTURED MEDICAL REPORT FOR CANCER IMAGING” filed on Oct. 5, 2023, the entire contents of which is hereby incorporated by reference in their entirety.

### TECHNICAL FIELD

The present disclosure relates generally to systems and methods for generating electronic medical reports in patients and, in some non-limiting embodiments, to systems and methods for generating electronic medical reports using positron emission tomography or single-photon emission computed tomography with computed tomography images.

### BACKGROUND

Medical imaging techniques, such as positron emission tomography (PET), single-photon emission computed tomography (SPECT), and computed tomography (CT), can be used to locate, diagnose, and stage cancer in patients. Physicians review and interpret medical images to generate reports that communicate findings to cancer care teams for clinical decision-making. However, generating comprehensive and structured medical reports from imaging data is challenging due to the complexity of integrating clinical information, imaging findings, and standardized staging classifications.

### SUMMARY

Methods and systems are described herein for novel uses and/or improvements to artificial intelligence applications. As one example, methods and systems are described herein for generating electronic medical reports using positron emission tomography or single-photon emission computed tomography with computed tomography images.

Existing systems for generating medical reports from cancer imaging data face significant technical limitations that prevent comprehensive and accurate report generation. From an architectural perspective, patient clinical data, imaging data, and laboratory results are typically stored across disparate database systems with different data formats and access protocols. For example, Electronic Health Record (EHR) systems, Picture Archiving and Communication Systems (PACS), and Radiology Information Systems (RIS) often operate as isolated data silos with heterogeneous database schemas, requiring separate authentication credentials, distinct application programming interfaces (APIs), and different data serialization formats. This fragmented data architecture creates substantial integration challenges, as each system may implement different security protocols, access control mechanisms, and data exchange standards that prevent automated aggregation of patient information into a unified report structure.

Furthermore, existing systems lack standardized data structures for medical report output, resulting in reports with inconsistent schemas that vary across institutions and individual practitioners. This absence of standardization creates downstream interoperability failures, as clinical decision support systems, treatment planning software, and other automated systems that consume these reports must be individually configured to parse each unique report format. For example, a downstream system designed to extract cancer staging information from a report generated at one institution may fail to correctly parse a report from another institution due to differences in field naming conventions, data type representations, or structural organization. The primary tumor, nodes, metastasis (TNM) classification is a widely accepted staging system for cancer, yet existing reporting systems lack automated mechanisms to extract and encode staging information in a machine-readable format, requiring manual data entry that introduces transcription errors and format inconsistencies. Additionally, existing artificial intelligence-based systems for cancer detection and segmentation operate in isolation from report generation pipelines, lacking the integration architecture necessary to automatically populate structured report templates with extracted imaging features, quantitative tumor characteristics, and staging classifications.

As a result of these architectural deficiencies, existing systems require multiple network requests across different database endpoints to retrieve the complete set of patient information necessary for clinical decision-making. Each query to a separate data source introduces network latency, increases bandwidth consumption, and requires separate authentication handshakes, resulting in inefficient resource utilization and increased system response times. The lack of a unified data model means that information retrieved from different sources cannot be automatically correlated without manual intervention, as patient identifiers, timestamp formats, and data field semantics may differ across systems. Moreover, existing systems lack predictive model integration capabilities that would enable automatic generation of prognostic or therapy-response predictions based on quantitative tumor characteristics extracted from imaging data. Without a processing pipeline that connects image segmentation outputs to clinical data repositories and predictive analytics engines, the computational resources required to generate comprehensive reports scale linearly with the number of manual data retrieval and integration steps, creating bottlenecks that limit system throughput and scalability.

To overcome these technical deficiencies, the methods and systems described herein implement an integrated data processing architecture that generates electronic medical reports for cancer imaging by automatically importing clinical patient data and digital imaging data through unified API interfaces, processing image data using predictive models to extract quantitative tumor characteristics, generating prognostic or therapy-response predictions, and populating a predefined template to produce a structured electronic medical report with a standardized schema. For example, the system addresses the architectural deficiencies of existing systems by implementing input components that establish secure connections to institution EHR databases and PACS/RIS environments, enabling automated data retrieval through standardized API protocols that eliminate the need for separate manual queries to disparate data sources. By consolidating data import, image processing, and report generation within a unified processing pipeline, the methods and systems described herein reduce network overhead associated with multiple cross-system queries, ensure data format consistency through standardized parsing and transformation operations, and produce machine-readable report outputs that enable seamless integration with downstream clinical decision support systems.

To do so, the system imports, based on a patient identifier for a patient, clinical patient data from a record database and digital imaging data comprising image data and acquisition metadata generated when imaging the patient. For example, the system may retrieve at least patient demographics, cancer history, laboratory-test results, and genetic markers in accordance with a secure application programming interface (API) from the record database, and retrieve a plurality of Digital Imaging and Communications in Medicine (DICOM) files associated with the image data and the acquisition metadata. The system implements data format validation and structure verification before passing data to processing components, ensuring compatibility with downstream processing stages, and eliminating format-related parsing failures. The system then processes the image data by executing a first predictive model to generate an output image representing segmentations of cancer lesions from non-cancerous tissue represented by the image data. For example, the first predictive model may generate an output image comprising a plurality of pixels, each pixel corresponding to tissue with cancer lesions or non-cancerous tissue. The system extracts quantitative tumor characteristics representing a cancer stage in accordance with a predefined staging classification based on the output image, including determining a tumor burden and a degree of expression of at least one tumor marker for every segmented lesion, and associating each lesion with an anatomical region within the image data. By implementing automated staging classification extraction that encodes cancer stage information in a standardized machine-readable format, the system enables downstream systems to consume staging data without requiring custom parsing logic for each report source.

The system then generates, by executing a second predictive model, at least one prognostic or therapy-response prediction for the patient based on the quantitative tumor characteristics and the clinical patient data. This integrated predictive model execution addresses the architectural gap in existing systems where image analysis components operate in isolation from clinical data repositories, preventing automated correlation of imaging features with patient history for prognostic assessment. The system generates an electronic medical report having a predetermined structure by populating a predefined template with the clinical patient data, the acquisition metadata, the cancer stage, the quantitative tumor characteristics, and the at least one prognostic or therapy-response prediction. By generating reports with a standardized schema that defines consistent field names, data types, and structural organization, the system enables interoperability with downstream clinical systems without requiring per-institution configuration. This architectural approach consolidates data retrieval operations into a single processing pipeline, reducing the number of network requests from multiple cross-system queries to a unified import operation, decreasing aggregate network latency, and enabling efficient caching of intermediate processing results. The standardized report output format ensures that clinical decision support systems, treatment planning software, and public health data aggregation systems can consume report data through a common parsing interface, eliminating the integration overhead associated with heterogeneous report formats.

In an embodiment, a system for generating electronic medical reports using positron emission tomography or single-photon emission computed tomography with computed tomography images is disclosed. The system can include one or more processors configured to import, based on a patient identifier for a patient, clinical patient data from a record database and digital image data including image data and acquisition metadata generated when imaging the patient. In some aspects, the one or more processors can be configured to process the image data by executing a first predictive model to generate an output image representing segmentations of cancer lesions from non-cancerous tissue represented by the image data. In some aspects, the one or more processors can be configured to extract quantitative tumor characteristics representing a cancer stage in accordance with a predefined staging classification based on the output image. In some aspects, the one or more processors can be configured to generate, by executing a second predictive model, at least one prognostic or therapy-response prediction for the patient based on the quantitative tumor characteristics and the clinical patient data. In some aspects, the one or more processors can be configured to generate an electronic medical report having a predetermined structure by populating a predefined template with the clinical patient data, the acquisition metadata, the cancer stage, the quantitative tumor characteristics and the at least one prognostic or therapy-response prediction.

In some aspects, the one or more processors can be configured to store the electronic medical report in an electronic data repository for access by authorized clinicians. In some aspects, in response to receiving a request for at least a portion of the electronic medical report, the one or more processors can be configured to compare a user identifier associated with the request to a predetermined user identifier assigned to the electronic medical report. In response to determining the user identifier matches the predetermined user identifier, the one or more processors can be configured to provide the electronic medical report to cause a display device associated with the predetermined user identifier to generate a user interface based on the electronic medical report.

In some aspects, the one or more processors configured to import the clinical patient data can be configured to retrieve at least patient demographics, cancer history, laboratory-test results, and genetic markers in accordance with a secure application programming interface (API) from the record database. In some aspects, the one or more processors configured to import the digital imaging data can be configured to retrieve a plurality of Digital Imaging and Communications in Medicine (DICOM) files associated with the image data and the acquisition metadata.

In some aspects, the first predictive model can be configured to generate an output image including a plurality of pixels, each pixel corresponding to tissue with cancer lesions or non-cancerous tissue.

In some aspects, the one or more processors configured to extract the quantitative tumor characteristics can be configured to, for every segmented lesion, determine a tumor burden and a degree of expression of at least one tumor marker. In some embodiments, the one or more processors can be configured to associate each lesion with an anatomical region within the image data.

In some aspects, the one or more processors can be configured to determine the cancer stage based on the tumor burden represented by each lesion of the anatomical region.

In some aspects, the one or more processors can be configured to determine an eligibility state for the patient based on the output image and the at least one prognostic or therapy-response prediction. In some aspects, the one or more processors configured to generate the electronic medical report can be configured to generate the electronic medical report based on the clinical patient data, the acquisition metadata, the cancer stage, the quantitative tumor characteristics the at least one prognostic or therapy-response prediction, and an indication of the eligibility state.

In another embodiment, a method for generating electronic medical reports using positron emission tomography or single-photon emission computed tomography with computed tomography images is disclosed. The method can include importing, by one or more processors and based on a patient identifier for a patient, clinical patient data from a record database and digital image data including image data and acquisition metadata generated when imaging the patient. In some aspects, the method can include processing, by the one or more processors, the image data by executing a first predictive model to generate an output image representing segmentations of cancer lesions from non-cancerous tissue represented by the image data. In some aspects, the method can include extracting, by the one or more processors, quantitative tumor characteristics representing a cancer stage in accordance with a predefined staging classification based on the output image. In some aspects, the method can include generating, by the one or more processors executing a second predictive model, at least one prognostic or therapy-response prediction for the patient based on the quantitative tumor characteristics and the clinical patient data. In some aspects, the method can include generating, by the one or more processors, an electronic medical report having a predetermined structure by populating a predefined template with the clinical patient data, the acquisition metadata, the cancer stage, the quantitative tumor characteristics and the at least one prognostic or therapy-response prediction.

In some aspects, the method can include storing, by the one or more processors, the electronic medical report in an electronic data repository for access by authorized clinicians. In some aspects, in response to receiving a request for at least a portion of the electronic medical report, the method can include comparing, by the one or more processors, a user identifier associated with the request to a predetermined user identifier assigned to the electronic medical report. In response to determining the user identifier matches the predetermined user identifier, the method can include providing, by the one or more processors, the electronic medical report to cause a display device associated with the predetermined user identifier to generate a user interface based on the electronic medical report.

In some aspects, importing the clinical patient data can include retrieving, by the one or more processors, at least patient demographics, cancer history, laboratory-test results, and genetic markers in accordance with a secure application programming interface (API) from the record database. In some aspects, importing the digital imaging data, by the one or more processors, can include retrieving a plurality of Digital Imaging and Communications in Medicine (DICOM) files associated with the image data and the acquisition metadata.

In some aspects, the first predictive model can be configured to generate an output image including a plurality of pixels, each pixel corresponding to tissue with cancer lesions or non-cancerous tissue.

In some aspects, extracting the quantitative tumor characteristics can include, for every segmented lesion, determining, by the one or more processors, a tumor burden and a degree of expression of at least one tumor marker. In some embodiments, the method can include associating, by the one or more processors, each lesion with an anatomical region within the image data.

In some aspects, the method can include determining, by the one or more processors, the cancer stage based on the tumor burden represented by each lesion of the anatomical region.

In some aspects, the method can include determining, by the one or more processors, an eligibility state for the patient based on the output image and the at least one prognostic or therapy-response prediction. In some aspects, generating the electronic medical report can include generating, by the one or more processors, the electronic medical report based on the clinical patient data, the acquisition metadata, the cancer stage, the quantitative tumor characteristics the at least one prognostic or therapy-response prediction, and an indication of the eligibility state.

In yet another embodiments, a non-transitory computer-readable medium is disclosed. The non-transitory computer-readable medium can store instructions thereon that, when executed by one or more processors, cause the one or more processors to import, based on a patient identifier for a patient, clinical patient data from a record database and digital image data including image data and acquisition metadata generated when imaging the patient. In some aspects, the instructions can cause the one or more processors to process the image data by executing a first predictive model to generate an output image representing segmentations of cancer lesions from non-cancerous tissue represented by the image data. In some aspects, the instructions can cause the one or more processors to extract quantitative tumor characteristics representing a cancer stage in accordance with a predefined staging classification based on the output image. In some aspects, the instructions can cause the one or more processors to generate, by executing a second predictive model, at least one prognostic or therapy-response prediction for the patient based on the quantitative tumor characteristics and the clinical patient data. In some aspects, the instructions can cause the one or more processors to generate an electronic medical report having a predetermined structure by populating a predefined template with the clinical patient data, the acquisition metadata, the cancer stage, the quantitative tumor characteristics and the at least one prognostic or therapy-response prediction.

In some aspects, the instructions can cause the one or more processors to store the electronic medical report in an electronic data repository for access by authorized clinicians. In some aspects, in response to receiving a request for at least a portion of the electronic medical report, the instructions can cause the one or more processors to compare a user identifier associated with the request to a predetermined user identifier assigned to the electronic medical report. In response to determining the user identifier matches the predetermined user identifier, the instructions can cause the one or more processors to provide the electronic medical report to cause a display device associated with the predetermined user identifier to generate a user interface based on the electronic medical report.

In some aspects, the instructions that cause the one or more processors to import the clinical patient data can cause the one or more processors to retrieve at least patient demographics, cancer history, laboratory-test results, and genetic markers in accordance with a secure application programming interface (API) from the record database. In some aspects, the instructions that cause the one or more processors to import the digital imaging data can cause the one or more processors to retrieve a plurality of Digital Imaging and Communications in Medicine (DICOM) files associated with the image data and the acquisition metadata.

In some aspects, the first predictive model can be configured to generate an output image including a plurality of pixels, each pixel corresponding to tissue with cancer lesions or non-cancerous tissue.

In some aspects, the instructions that cause the one or more processors to extract the quantitative tumor characteristics can cause the one or more processors to, for every segmented lesion, determine a tumor burden and a degree of expression of at least one tumor marker. In some embodiments, the instructions can cause the one or more processors to associate each lesion with an anatomical region within the image data.

In some aspects, the instructions can cause the one or more processors to determine the cancer stage based on the tumor burden represented by each lesion of the anatomical region.

In some aspects, the instructions can cause the one or more processors to determine an eligibility state for the patient based on the output image and the at least one prognostic or therapy-response prediction. In some aspects, the instructions that cause the one or more processors to generate the electronic medical report can cause the one or more processors to generate the electronic medical report based on the clinical patient data, the acquisition metadata, the cancer stage, the quantitative tumor characteristics the at least one prognostic or therapy-response prediction, and an indication of the eligibility state.

### BRIEF DESCRIPTION OF THE DRAWINGS

Non-limiting embodiments of the present disclosure are described by way of example with reference to the accompanying figures, which are schematic and are not intended to be drawn to scale. Unless indicated as representing the background art, the figures represent aspects of the disclosure.

FIG. 1 illustrates a diagram of a system for generating electronic medical reports using positron emission tomography or single-photon emission computed tomography with computed tomography images, in accordance with one or more embodiments.

FIG. 2 illustrates a resident device involved in generating electronic medical reports using positron emission tomography or single-photon emission computed tomography with computed tomography images, in accordance with one or more embodiments.

FIG. 3 illustrates a cloud service involved in generating electronic medical reports using positron emission tomography or single-photon emission computed tomography with computed tomography images, in accordance with one or more embodiments.

FIG. 4 illustrates an artificial intelligence model that is configured to generate outputs, in accordance with one or more embodiments.

FIG. 5 illustrates an electronic medical report generated using positron emission tomography or single-photon emission computed tomography with computed tomography images, in accordance with one or more embodiments.

FIG. 6 shows a flowchart of the steps involved in generating electronic medical reports using positron emission tomography or single-photon emission computed tomography with computed tomography images, in accordance with one or more embodiments.

### DETAILED DESCRIPTION

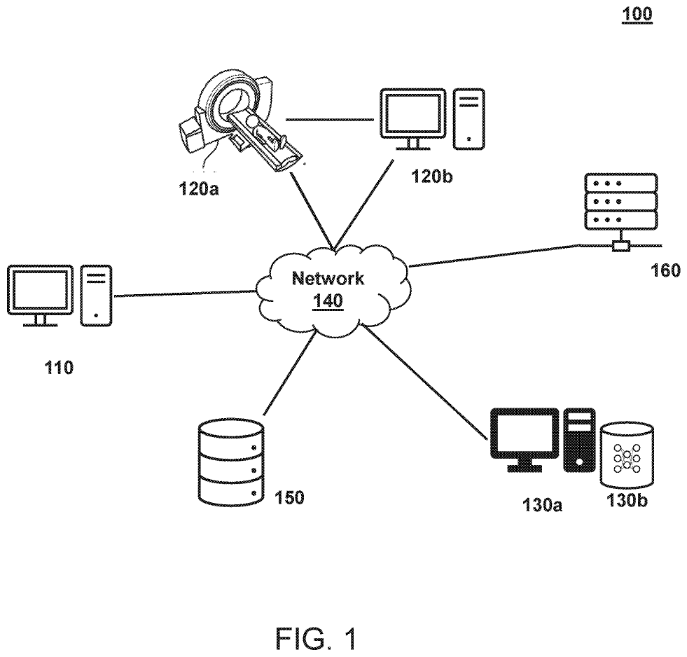

FIG. 1 illustrates a diagram of a system for generating electronic medical reports using positron emission tomography or single-photon emission computed tomography with computed tomography images, in accordance with one or more embodiments. For example, FIG. 1 illustrates components of an environment 100 for generating electronic medical reports using positron emission tomography or single-photon emission computed tomography with computed tomography images, according to an embodiment. The environment 100 can include a client device 110, an imaging device 120a, a clinician workstation 120b, an analytics server 130a, an analytics database 130b, a patient database 150, and a public health server 160. Various components depicted in FIG. 1 can belong to a treatment clinic involved in diagnosing and treating diseases such as cancers described herein. The environment 100 is not confined to the components described herein and can include additional or other components, not shown for brevity, which are configured to be considered within the scope of the embodiments described herein.

The above-mentioned components can be connected to each other through a network 140. Examples of the network 140 can include, but are not limited to, private or public local-area-networks (LAN), wireless LAN (WLAN) networks, metropolitan area networks (MAN), wide-area networks (WAN), and the Internet. The network 140 can include wired and/or wireless communications according to one or more standards and/or via one or more transport mediums. The communication over the network 140 can be performed in accordance with various communication protocols such as Transmission Control Protocol and Internet Protocol (TCP/IP), User Datagram Protocol (UDP), and IEEE communication protocols. In one example, the network 140 can include wireless communications according to Bluetooth specification sets or another standard or proprietary wireless communication protocol. In another example, the network 140 can also include communications over a cellular network, including, e.g., a GSM (Global System for Mobile Communications), CDMA (Code Division Multiple Access), and EDGE (Enhanced Data for Global Evolution) network.

The client device 110 can be any computing device comprising a processor and non-transitory machine-readable storage capable of executing the various tasks and processes described herein. The client device 110 can employ various processors such as central processing units (CPU) and graphics processing unit (GPU), among others. Non-limiting examples of such computing devices can include workstation computers, laptop computers, server computers, and the like. While the environment 100 includes a single client device 110, the client device 110 can include any number of computing devices operating in a distributed computing environment, such as a cloud environment. In some embodiments, the client device 110 can be associated with a clinician (e.g., an oncologist and/or the like) that is screening and/or treating one or more patients with one or more diseases such as, for example, cancers including prostate cancers and/or the like.

The imaging device 120a can be a diagnostic imaging device or a treatment delivery device. For example, the imaging device 120a can include one or more computed tomography (CT) scanners, positron emission tomography (PET) scanners, a combination PET/CT scanner that is configured to either simultaneously or in rapid succession generate CT images and PET images of a patient, and/or the like. In examples, the imaging device 120a can include SPECT/CT scanners and the analysis described herein can be performed using SPECT images. In some embodiments, the imaging device 120a can be configured to generate one or more CT and/or one or more functional images such as PET images of a patient as described herein. In some embodiments, the imaging device 120a can include one or more magnetic resonance imaging (MRI) scanners, scintigraphy devices, or other diagnostic imaging equipment. For example, the imaging device 120a can include an MRI scanner configured to generate anatomical images of soft tissue structures, or a scintigraphy device configured to detect gamma radiation emitted by radiopharmaceuticals administered to the patient.

The imaging device 120a can be configured to communicate with various sensors (not explicitly illustrated) that monitor a patient's external biological signals. Non-limiting examples of the sensors can include 3D surfacing mechanisms and optical (or other) sensors configured to monitor the movements by the patient (e.g., in response to respiration by the patient when breathing). In some embodiments, the imaging device 120a can be associated with a clinician that is the same as, or similar to, the clinician associated with the client device 110 and/or any other suitable technician that can operate the imaging device 120a.

The imaging device 120a can be associated with (e.g., interconnected with) a clinician workstation 120b and configured to control operation of the imaging device 120a during operation of the imaging device 120a when generating one or more images (e.g., CT and/or PET images) of a patient. For example, the clinician workstation 120b can be any computing device comprising a processor and non-transitory machine-readable storage capable of executing the various tasks and processes described herein. The clinician workstation 120b can employ various processors such as central processing units (CPU) and graphics processing unit (GPU), among others. Non-limiting examples of such computing devices can include workstation computers, laptop computers, server computers, and the like. In some embodiments, the clinician workstation 120b can be associated with a clinician that is the same as, or similar to, the clinician associated with the client device 110 and/or any other suitable technician that can operate the imaging device 120a.

Clinician workstation 120b may generate one or more image acquisition files when imaging the patient via imaging device 120a. For example, the clinician workstation 120b may generate image acquisition files that include image data and acquisition metadata associated with the imaging procedure. Image data may include any digital representation of medical images captured during the imaging procedure, such as cross-sectional anatomical images, functional images showing metabolic activity, fused images combining anatomical and functional information, or reconstructed three-dimensional volumetric data. Acquisition metadata may include any information describing the technical parameters and circumstances of the image acquisition process, such as scanner configuration settings, patient positioning information, timing parameters, contrast or radiopharmaceutical administration details, reconstruction algorithms applied, or quality control measurements. In some embodiments, the image data and acquisition metadata may be stored in Digital Imaging and Communications in Medicine (DICOM) files that include image data and acquisition metadata associated with the imaging procedure. The acquisition metadata may include information such as the type of scanner used for image acquisition, the type and amount of radiopharmaceutical injected, the type and amount of contrast agent injected, time of the scanning procedure, the location of the scanning procedure, patient information (e.g., patient name, date of birth, age, sex, weight, height, patient identification number, referring physician name, institution name, etc.), or other information. The clinician workstation 120b may store the image acquisition files in the patient database 150 along with other patient information, such as patient clinical data (e.g., patient demographics, cancer history, laboratory-test results, and genetic markers) and patient identifiers that associate the image acquisition files with a particular patient. By storing the image acquisition files in the patient database 150, the analytics server 130a and other components of the environment 100 may retrieve the image acquisition files for subsequent processing and analysis.

The analytics server 130a can be any computing device comprising a processor and non-transitory machine-readable storage capable of executing the various tasks and processes described herein. The analytics server 130a can employ various processors such as central processing units (CPU) and graphics processing unit (GPU), among others. Non-limiting examples of such computing devices can include workstation computers, laptop computers, server computers, and the like. While the environment 100 includes a single analytics server 130a, the environment 100 can include any number of analytics servers operating in a distributed computing environment, such as a cloud environment.

The analytics server 130a can generate and display an electronic platform configured to receive and process inputs from clinicians and/or PET/CT images (e.g., generated by the imaging device 120a), and perform one or more of the operations described herein to identify and diagnose the presence of one or more forms of cancer in patients. In some embodiments, the electronic platform generates a graphical user interface (GUI) that is displayed by display devices of the analytics server 130a and/or the client device 110. An example of the electronic platform generated and hosted by the analytics server 130a can include a web-based application or a website configured to be displayed on one or more of the devices of the environment 100.

The analytics database 130b can be any computing device comprising a processor and non-transitory machine-readable storage capable of executing the various tasks and processes described herein. For example, the analytics database 130b can represent various computing devices that contain, retrieve, and/or access data associated with the client device 110, the imaging device 120a and/or the analytics server 130a, such as data associated with current and/or previously monitored patients (e.g., CT images, PET images, SPECT images, tumor locations, and/or the like). For instance, the analytics server 130a can obtain data associated with operation of the imaging device 120a and process the data. The analytics server 130a can then generate a dataset and/or data associated with one or more patients, and use the dataset and/or data to train one or more models and/or model ensembles as described herein.

The analytics server 130a and the analytics database 130b may be hosted in a cloud computing environment to provide scalable processing capabilities and centralized data management. For example, the analytics server 130a and the analytics database 130b may be implemented as part of a cloud-based system that receives patient data from one or more resident devices and may provide data back to the one or more resident devices. For example, resident devices may include client devices 110, imaging devices 120a, clinician workstations 120b, or other devices located at different healthcare facilities. By hosting the analytics server 130a and the analytics database 130b in a cloud computing environment, the environment 100 can leverage networking gains of scale and scope, enable federated learning strategies across healthcare facilities, and provide distributed updates to artificial intelligence models deployed at resident parts.

The patient database 150 can be any computing device comprising a processor and non-transitory machine-readable storage capable of storing patient-related (e.g., user-related) data. For example, the patient database 150 can represent a local database that is deployed within a healthcare institution. In some embodiments, the patient database 150 can store various types of patient data. The patient database 150 can store clinical patient data including patient demographics, clinical history, cancer history, laboratory test results, and genetic markers. The patient database 150 can also store digital imaging data including image data and acquisition metadata generated when imaging patients, such as Digital Imaging and Communications in Medicine (DICOM) files accessed from an institution Picture Archiving and Communication System (PACS) within a Radiology Information System (RIS) environment. In some embodiments, the patient database 150 can store patient identifiers that associate the clinical patient data and digital imaging data with particular patients.

The patient database 150 may store information transmitted from the client device 110, the imaging device 120a, the clinician workstation 120b, or other devices connected to the network 140. For example, the client device 110 may transmit patient clinical data entered by a clinician to the patient database 150 for storage and subsequent retrieval. The imaging device 120a may transmit image acquisition files including image data and acquisition metadata to the patient database 150 after imaging a patient. For example, the clinician workstation 120b may transmit DICOM files and associated metadata to the patient database 150 following an imaging procedure. In some embodiments, the client device 110 may access the patient database 150 via the network 140 to retrieve clinical patient data and digital imaging data for review by a clinician. The imaging device 120a may access the patient database 150 to retrieve patient identifiers and prior imaging data to associate with new imaging procedures. The clinician workstation 120b may access the patient database 150 to retrieve patient clinical data and prior imaging data for display during image acquisition and review. Each of these devices may establish secure connections with the patient database 150 using encrypted communication protocols and authentication mechanisms to ensure privacy and accuracy of patient data during transmission and retrieval.

In some implementations involving prostate cancer imaging, the patient database 150 can store cancer history data such as time of diagnosis of prostate cancer and serum PSA value at time of diagnosis, laboratory test results such as serum prostate-specific antigen (PSA) values, previous therapy information including surgery (radical prostatectomy), chemotherapy (e.g., docetaxel, cabazitaxel), and hormonal therapy (e.g., ADT, abiraterone, enzalutamide), and pathology results such as Gleason score at diagnosis or genetic mutation information (e.g., ATM and TP53 mutations). The patient database 150 can further store technical information extracted from DICOM metadata including the type of scanner used for image acquisition, the type and amount of radiopharmaceutical injected (e.g., 68Ga-PSMA-11, 18F-DCFPyL, 18F-rhPSMA-7.3), the type and amount of contrast agent injected, time of the scanning procedure, and the location of the scanning procedure.

The patient database 150 may be configured to transmit data to and receive data from the analytics database 130b via the network 140. For example, the patient database 150 may establish a secure connection with the analytics database 130b using encrypted communication protocols to ensure privacy and accuracy of patient data during transmission. The patient database 150 may transmit clinical patient data, digital imaging data, and patient identifiers to the analytics database 130b for processing by the analytics server 130a. In some embodiments, the patient database 150 may receive updated patient data, processed results, or artificial intelligence model outputs from the analytics database 130b. The communication between the patient database 150 and the analytics database 130b may be managed using application programming interface (API) systems that facilitate automatic data import and export. Security measures implemented during data transmission may include data encryption, authentication protocols, and access control mechanisms to guarantee that patient data is protected in accordance with applicable privacy requirements. By establishing this secure communication pathway between the patient database 150 and the analytics database 130b, the environment 100 may enable centralized data analysis while maintaining data security at the healthcare institution level.

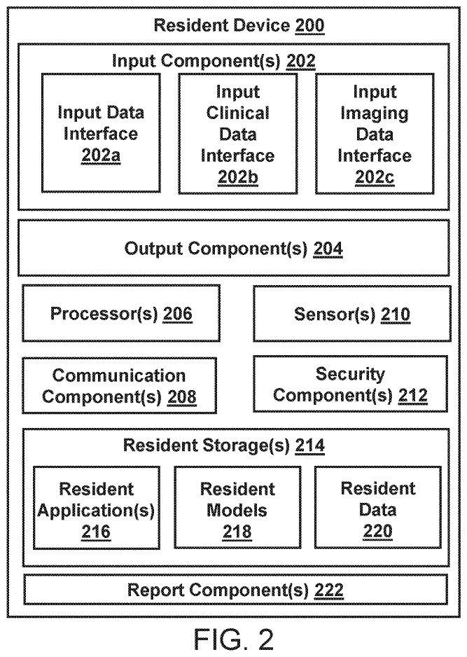

FIG. 2 illustrates a resident device involved in generating electronic medical reports using positron emission tomography or single-photon emission computed tomography with computed tomography images generating electronic medical reports, in accordance with one or more embodiments. For example, FIG. 2 shows a resident device 200 involved in generating electronic medical reports positron emission tomography or single-photon emission computed tomography with computed tomography images generate electronic medical reports. The resident device 200 includes input components 202, output components 204, processors 206, communication components 208, sensors 210, security components 212, resident storage 214, and report components 222. The resident storage 214 further includes resident applications 216, resident models 218, and resident data 220. The input components 202 include an input data interface 202a, an input clinical data interface 202b, and an input imaging data interface 202c.

In some embodiments, resident device 200 may correspond to any computing device deployed at a healthcare institution that is configured to interact with patient data. For example, the healthcare institution may include a hospital, a medical clinic, an imaging center, a cancer treatment facility, an outpatient diagnostic center, or any other facility where medical imaging procedures are performed and interpreted. The resident device 200 may be implemented as any of the devices described in connection with FIG. 1, including client device 110, imaging device 120a, or clinician workstation 120b. In some embodiments, the resident device 200 may correspond to client device 110. For example, a user such as a clinician, oncologist, or patient may use a workstation computer (e.g., client device 110) to perform healthcare-related tasks, including but not limited to accessing patient data, viewing medical reports, inputting clinical information, or reviewing imaging results. In some embodiments, the resident device 200 may correspond to imaging device 120a. For example, an imaging scanner may include integrated processing capabilities for performing various data processing and analysis functions (e.g., performing medical imaging, storing medical imaging data, storing acquisition data associated with medical images, etc.). In some embodiments, the resident device 200 may correspond to clinician workstation 120b. For example a technician, radiologist, or other healthcare professional may use a dedicated workstation interconnected with the imaging device 120a to perform clinical workflows, perform medical imaging tasks, access patient records, or interact with healthcare systems. In some embodiments, the resident device 200 may be a standalone computing device separate from the devices depicted in FIG. 1 but connected to the network 140 and configured to retrieve or store patient data to and from the patient database 150, communicate with the analytics server 130a, or perform other operations, in accordance with one or more embodiments.

The input components 202 may be configured to receive data for processing by the resident device 200. The input components 202 may include one or more user interfaces for accepting information from users, such as graphical user interfaces (GUIs) that display input fields, dropdown menus, checkboxes, and other interactive elements for data entry. The input components 202 may also include various input mechanisms such as keyboards, mice, touchscreens, styluses, microphones for voice input, barcode scanners, or other computer peripherals configured to accept inputs. In some embodiments, the input components 202 may include web-based interfaces accessible through a browser, dedicated software applications with custom input forms, or mobile device interfaces for portable data entry. The input components 202 may receive data from users such as clinicians, radiologists, technicians, or administrative personnel who manually enter patient information, clinical observations, clinical data, or other relevant data. The input components 202 may also receive data from external systems via communication components 208, such as automated data feeds, application programming interfaces (APIs), or direct database connections.

The input data interface 202a may be responsible for receiving data at a user interface of the resident device 200. For example, the input data interface 202a may receive patient data entered manually by a user such as a radiologist. The input data interface 202a may perform data validation and format verification operations before storing the data in resident storage 214 (e.g., resident data 220). For example, the input data interface 202a may extract input data from user-provided entries, determine a format type associated with the extracted input data, and compare the determined format type against a predefined format specification corresponding to the data field. To perform this comparison, the input data interface 202a may retrieve format specification data from the resident data 220 of resident storage 214. The format specification data may indicate expected data structures, encoding requirements, and validation rules for each data field type. The format specification data may include schema definitions that specify required field lengths, acceptable character sets, mandatory field relationships, and hierarchical data organization patterns for different categories of clinical and imaging data. The input data interface 202a may parse the extracted input data to identify structural elements such as field delimiters, data type indicators, and encoding schemes. The input data interface 202a may then compare each identified structural element against the corresponding validation rule specified in the format specification data. For example, the input data interface 202a may verify that numerical fields contain only numeric characters within specified ranges, that date fields conform to expected date format patterns, that required fields are populated with non-null values, and that relational constraints between dependent fields are satisfied. The input data interface 202a may execute a series of validation checks that evaluate the extracted input data against each applicable rule in the format specification data, generating a validation result for each check. In response to determining that the extracted input data satisfies the applicable validation rules and conforms to the predefined format specifications, the input data interface 202a may store the validated data in the resident data 220 of resident storage 214 for subsequent processing by the processors 206. By storing only data that has been verified to conform to the format specifications, the input data interface 202a ensures that downstream processing components receive properly formatted data, thereby reducing computational overhead associated with error handling and preventing data corruption that could result from processing malformed inputs.

In some embodiments, the input data interface 202a may perform syntactic validation by analyzing character sequences within the extracted input data to verify compliance with expected patterns. For example, the input data interface 202a may verify that date fields conform to a standardized date format or that numerical values fall within acceptable ranges. The input data interface 202a may further perform semantic validation by cross-referencing the extracted input data against reference data stored in the resident data 220 to verify logical consistency. For example, the input data interface 202a may confirm that patient identifiers correspond to existing patient records or that anatomical region codes match predefined anatomical classification schemas. To perform this semantic validation, the input data interface 202a may retrieve reference data from the resident data 220 of resident storage 214. The reference data may indicate valid patient identifiers, acceptable anatomical region codes, and logical relationships between data fields. The input data interface 202a may compare each element of the extracted input data against the corresponding reference data to identify malformed or incompatible data entries before the data propagates to downstream processing components. In response to determining that the extracted input data satisfies the syntactic and semantic validation criteria, the input data interface 202a may store the validated data in the resident data 220 of resident storage 214 for subsequent processing by the processors 206. By storing only data that has been verified to conform to the format specifications and logical consistency requirements, the input data interface 202a ensures that downstream processing components receive properly formatted and logically consistent data, thereby reducing computational overhead associated with error handling and preventing data corruption that could result from processing malformed inputs. In response to determining that the extracted input data fails to satisfy the predefined format specification or validation criteria, the input data interface 202a may generate an error indication and prompt the user to correct the input data before proceeding.

The input clinical data interface 202b may be responsible for importing patient data to one or more databases or storages described herein. For example, the input clinical data interface 202b may import clinical patient data from institution electronic health record (EHR) databases. The input clinical data interface 202b may retrieve clinical patient data from the resident data 220 of resident storage 214, which in some embodiments, may correspond to a local storage instance of patient data maintained on the resident device 200. In some embodiments, the input clinical data interface 202b may retrieve clinical patient data from external databases accessible via the network 140 through communication components 208, such as the patient database 150 or the analytics database 130b. The input clinical data interface 202b may establish secure connections with these databases using encrypted communication protocols and authentication mechanisms to retrieve clinical patient data. The clinical patient data may include various types of patient information relevant to cancer diagnosis, treatment, and management. For example, the clinical patient data may include patient demographics such as age, gender, and comorbidities. The clinical patient data may include cancer history such as time of diagnosis, initial diagnosis details, pathology results, cancer stage at diagnosis, and treatment history. The clinical patient data may include laboratory test results such as tumor markers, serum biomarker values, and complete blood count results. The clinical patient data may include genetic markers and genomic information such as gene mutations, epigenomic data, and molecular profiling results. The clinical patient data may include previous therapy information such as surgical procedures, chemotherapy regimens, hormonal therapy, immunotherapy, radiation therapy, and targeted therapy. The clinical patient data may include pathology results such as histological grade, receptor status, and tissue biopsy findings. The clinical patient data may include clinical state information indicating the current disease status or progression stage. The clinical patient data may include clinical indications for the imaging procedure such as initial staging, re-staging, treatment response evaluation, or surveillance. The clinical patient data may include any other patient-related information stored in electronic health records that is relevant to the generation of the electronic medical report for cancer imaging.

Input clinical data interface 202b may import patient data automatically using application programming interface (API) systems. In some embodiments, the input clinical data interface 202b may retrieve clinical patient data in accordance with a secure application programming interface (API) from the record database. Data recognition and extraction may be performed using natural language processing and machine learning techniques, depending on the type of data being extracted. By automatically importing clinical patient data from institution EHR databases and other data sources, the input clinical data interface 202b eliminates the ineffective, time-consuming, and error-prone manual review of Electronic Health Records that existing systems require radiologists to perform, thereby reducing clinician workload and decreasing the likelihood of transcription errors during report generation.

Similar to input data interface 202a, the input clinical data interface 202b may perform data format validation and completeness verification before storing the clinical patient data in the resident data 220 of resident storage 214 (or other storages or databases as described herein). For example, the input clinical data interface 202b may determine whether retrieved clinical patient data conforms to a predefined format specification and whether required data fields are populated with valid values. In response to determining that the clinical patient data is in an incorrect format, is missing required fields, or is not available from the record database, the input clinical data interface 202b may automatically invoke the input data interface 202a to accept manually entered data from a user such as a radiologist. For example, the input clinical data interface 202b may transmit an invocation message to the input data interface 202a, wherein the invocation message includes a field identifier indicating the specific data field that requires manual input, a data type specification indicating the expected format for the data field, and a validation rule set indicating the constraints that the manually entered data must satisfy.

In response to receiving the invocation message, the input data interface 202a may parse the field identifier, data type specification, and validation rule set from the invocation message, and generate a user interface element corresponding to the identified data field. For example, the input data interface 202a may render an input form on a display device associated with the resident device 200, wherein the input form includes a text field, dropdown menu, or other input element configured to accept user input for the identified data field. The input data interface 202a may display a prompt message indicating the type of information required and any formatting constraints specified in the validation rule set. Upon receiving user input via the rendered input element, the input data interface 202a may validate the entered data against the validation rule set received in the invocation message, and in response to determining the entered data satisfies the validation rules, transmit the validated data to the input clinical data interface 202b. The input clinical data interface 202b may then receive the manually entered data from the input data interface 202a, perform additional validation on the manually entered data to verify format compliance and logical consistency with other clinical patient data, and store the validated clinical patient data in the resident data 220 of resident storage 214 for subsequent processing by the processors 206. By automatically invoking the input data interface 202a when automated data retrieval fails or returns incomplete data, the input clinical data interface 202b ensures that the clinical patient data required for generating the electronic medical report is complete and properly formatted, thereby preventing downstream processing failures and ensuring report accuracy.

The input imaging data interface 202c may be responsible for importing digital imaging data comprising image data and acquisition metadata generated when imaging the patient. For example, the input imaging data interface 202c may input technical information that describes the process of medical image acquisition. The input imaging data interface 202c may retrieve digital imaging data from the resident data 220 of resident storage 214, which may store locally cached imaging files, or from external systems such as the patient database 150 or the analytics database 130b accessible via the network 140. The input imaging data interface 202c may extract information from metadata of Digital Imaging and Communications in Medicine (DICOM) files. In some embodiments, the input imaging data interface 202c may retrieve a plurality of Digital Imaging and Communications in Medicine (DICOM) files associated with the image data and the acquisition metadata. Sets of DICOM files may be accessed from an institution Picture Archiving and Communication System (PACS) within a Radiology Information System (RIS) environment. Information may be extracted automatically from DICOM metadata and may include the type of scanner used for image acquisition, the type and amount of radiopharmaceutical injected, the type and amount of contrast agent injected, time of the scanning procedure, and the location of the scanning procedure, or whether a diuretic was administered for the imaging procedure. The input imaging data interface 202c may perform a check for DICOM file format compliance and verify that required acquisition metadata fields are present before storing the digital imaging data in the resident data 220 of resident storage 214 for processing by the processors 206. By automatically extracting technical information from DICOM metadata, the input imaging data interface 202c eliminates the ineffective and time-consuming manual review of Radiology Information Systems that existing systems require radiologists to perform when documenting image acquisition parameters in medical reports.

The output components 204 may be configured to provide processed information and generated reports from the resident device 200. The output components 204 may include one or more output mechanisms for presenting information to users, such as display devices, speakers, haptic engines, printers, or other computer peripherals configured to provide outputs. In some embodiments, the output components 204 may include display output components configured to render visual information on screens, monitors, or other display devices. The display output components may generate graphical user interfaces (GUIs) that present electronic reports, including medical reports, imaging reports, diagnostic summaries, and other clinical documentation. The output components 204 may also include audio output components such as speakers configured to provide audible notifications, alerts, or voice-based information to users. The output components 204 may further include haptic output components such as haptic engines configured to provide tactile feedback to users through vibrations or other physical sensations. In some embodiments, the output components 204 may include printing output components configured to generate physical copies of electronic reports and other documents.

The output components 204 may receive information from the processors 206 and the resident storage 214, integrate the information in pertinent sections, and automatically generate a standardized medical report for display or transmission. For example, the output components 204 may render electronic medical reports on a display device associated with the resident device 200, enabling clinicians to review imaging findings, cancer staging information, and prognostic predictions. The medical report may integrate sections including clinical history, technical information about the process of image acquisition, oncological findings summarizing cancer stage in a standardized way following the TNM classification system, descriptive findings providing detailed description of cancer including anatomical localization, treatment response evaluation, eligibility status for therapy, patient prognosis, or other information.

The processors 206 may include any hardware or software processors configured to execute operations of the resident device 200. For example, the processors 206 may include central processing units (CPUs), graphics processing units (GPUs), application-specific integrated circuits (ASICs), field-programmable gate arrays (FPGAs), digital signal processors (DSPs), or any combination thereof. The processors 206 may be configured to process data within the resident device 200, where data from the input components 202, the output components 204, the communication components 208, the sensors 210, the security components 212, and the resident storage 214 may be received, transformed, and redirected through the processors 206. The processors 206 may interact with one or more components of the resident device 200 to coordinate data flow and execute computational tasks. The processors 206 may execute applications stored in the resident applications 216 to perform medical report generation functions and other operations. The processors 206 may execute artificial intelligence models retrieved from the resident models 218 to perform tumor detection, segmentation, classification on medical images, or other operations in accordance with one or more embodiments. The processors 206 may process various types of data including input data received from the input components 202, output data generated for the output components 204, patient data stored in the resident data 220, and intermediate data generated during processing operations. The processors 206 may process image data by executing predictive models stored in the resident models 218 to generate segmentations of cancer lesions. The processors 206 may further extract quantitative tumor characteristics based on the segmentations, including determining a tumor burden and a degree of expression of at least one tumor marker for every segmented lesion, and associating each lesion with an anatomical region within the image data to determine a cancer stage in accordance with a predefined staging classification such as the TNM classification system.

The communication components 208 may facilitate data transmission between the resident device 200's components, external components, or other devices and systems. For example, the communication components 208 may establish and manage network connections with other computing devices, servers, databases, and cloud-based services via the network 140. The communication components 208 may implement various communication protocols and interfaces to enable data exchange, including application programming interfaces (APIs), secure application programming interfaces (secure APIs), representational state transfer (REST) interfaces, and other standardized or proprietary communication protocols. In some embodiments, the communication components 208 may implement encrypted communication channels using transport layer security (TLS), secure sockets layer (SSL), or other encryption protocols to protect data during transmission. The communication components 208 may also implement authentication mechanisms, such as token-based authentication, certificate-based authentication, or multi-factor authentication, to verify the identity of external systems before establishing data connections. The communication components 208 may manage communication sessions with institution databases such as the patient database 150, cloud-based services such as the analytics server 130a and the analytics database 130b, and public health systems such as the public health server 160. In some embodiments, the communication components 208 may include network interface controllers, wireless communication modules, or other hardware components configured to transmit and receive data packets over wired or wireless network connections.

In some embodiments, the processors 206 may generate one or more metrics for evaluating treatment response in prostate cancer patients based on standardized criteria. For example, processors 206 may generate metrics based on criteria such as the Prostate Cancer Working Group 3 (PCWG3) criteria, the Prostate Cancer Working Group 4 (PCWG4) criteria, PSMA PET/CT Progression (PPP) criteria, or Response Evaluation Criteria in PSMA Imaging Prostate (RECIP) in conjunction with patient data (e.g., stored in the one or more storages described herein).

For example, the processors 206 may calculate PSA-based metrics including PSA response (a confirmed ≥50% decline from baseline) and PSA progression (a confirmed ≥25% increase and ≥2 ng/mL rise from nadir), as well as radiographic metrics based on RECIST 1.1 for soft-tissue lesions and the “2+2 rule” for bone metastases requiring confirmation of new lesions on subsequent scans. The processors 206 may generate response classifications such as complete response, partial response, stable disease, or progressive disease based on changes in lesion size, number, or metabolic activity between imaging studies. The PCWG4 criteria extend these calculations to accommodate PSMA PET imaging modalities, circulating tumor DNA markers, and composite endpoints combining PSA, imaging, and biomarker assessments. By implementing these standardized treatment response metrics, the processors 206 enable consistent and reproducible evaluation of therapy efficacy across imaging studies for inclusion in the electronic medical report.

In some embodiments, the processors 206 may generate metrics based on PSMA PET Progression (PPP) criteria or Response Evaluation Criteria in PSMA Imaging Prostate (RECIP) to evaluate disease progression and treatment response on PSMA PET imaging. The PPP criteria define progression on PSMA PET when new PSMA-avid lesions appear beyond physiologic distribution, when previously PSMA-positive lesions increase in size, intensity, or number, or when a worsening disease pattern is confirmed on follow-up scan. The PPP criteria address limitations of standard RECIST criteria for bone metastases, which are frequent in prostate cancer, by providing rules specifically designed for PSMA-targeted PET that detects microscopic changes earlier than CT or bone scan. The RECIP criteria provide a formalized quantitative system for assessing response or progression on PSMA PET, using measurable changes in the number of PSMA-avid lesions and total lesion uptake to classify response categories including complete molecular response, partial molecular response, stable molecular disease, and progressive molecular disease. While staging classifications such as PROMISE and miTNM standardize how disease is staged at a single timepoint, RECIP defines how changes are evaluated between timepoints for longitudinal assessment. By implementing PPP and RECIP metrics, the processors 206 enable molecular imaging-based response evaluation that captures disease changes detectable on PSMA PET imaging for inclusion in the electronic medical report.

The sensors 210 may include any sensor associated with a resident device 200. For example, the sensors 210 may include image sensors such as cameras for user authentication or document scanning, ambient light sensors, proximity sensors, accelerometers, gyroscopes, magnetometers, barometric pressure sensors, temperature sensors, humidity sensors, fingerprint sensors, microphones, global infrared sensors, heart rate or photoplethysmography (PPG) sensors, medical device sensors, or other sensors. In some embodiments, the sensors 210 may provide input data to the processors 206 for use in conjunction with the medical imaging and report generation processes described herein. For example, sensors 210 may be used to capturing images of physical documents for optical character recognition, obtain patient health data, or be used for other purposes, in accordance with one or more embodiments.

The security components 212 may implement data security measures within the resident device 200. For example, the security components 212 may be responsible for data encryption, access control enforcement, and other cyber-defense strategies to guarantee privacy and accuracy of data within the resident device 200. The security components 212 may implement encryption protocols for data at rest stored in the resident storage 214 and data in transit communicated via the communication components 208. For example, the security components 212 may encrypt patient clinical data, digital imaging data, and electronic medical reports using cryptographic algorithms such as Advanced Encryption Standard (AES) or other industry-standard encryption methods. The security components 212 may also implement access control mechanisms that restrict access to patient data based on user roles, permissions, and authentication credentials. For example, the security components 212 may verify user identities through multi-factor authentication before granting access to sensitive patient information or allowing modifications to electronic medical reports. The security components 212 may maintain audit logs that record access attempts, data modifications, and system events to enable security monitoring and compliance verification. The security components 212 may further implement intrusion detection and prevention capabilities to identify and respond to unauthorized access attempts or malicious activities targeting the resident device 200. By implementing these security measures, the security components 212 may ensure that patient data processed by the resident device 200 is protected in accordance with applicable privacy requirements, including healthcare data protection regulations and institutional security policies.

The resident storage 214 may contain resident applications 216, resident models 218, and resident data 220. The resident applications 216 may store software applications that execute on the processors 206. For example, the resident applications 216 may include software applications that aid in report generation, web browsers, data management utilities, or other applications that execute on the processors 206 to support the operations of the resident device 200. The resident models 218 may store configured (e.g., pre-trained) artificial intelligence models, machine learning models, predictive models, statistical models, or other models. For example, the resident models may be used for tumor detection, segmentation, and classification on medical images. For example, the resident models 218 may include a first predictive model configured to process image data and generate an output image representing segmentations of cancer lesions from non-cancerous tissue, and a second predictive model configured to generate prognostic or therapy-response predictions based on quantitative tumor characteristics and clinical patient data.

The resident data 220 may store various types of data used by the resident device 200 in generating electronic medical reports. For example, the resident data 220 may store clinical patient data imported from record databases, including patient demographics, cancer history, laboratory test results, and genetic markers. The resident data 220 may store digital imaging data comprising image data and acquisition metadata generated when imaging patients, such as Digital Imaging and Communications in Medicine (DICOM) files retrieved from institution Picture Archiving and Communication Systems. The resident data 220 may store format specification data indicating expected data structures, encoding requirements, and validation rules for data validation operations performed by the input data interface 202a. The resident data 220 may store reference data for semantic validation, including valid patient identifiers, acceptable anatomical region codes, and logical relationships between data fields. The resident data 220 may store predefined templates used for generating electronic medical reports having a predetermined structure. The resident data 220 may store intermediate processing results generated during image segmentation, quantitative tumor characteristic extraction, and prognostic or therapy-response prediction operations. The resident data 220 may store generated electronic medical reports populated with clinical patient data, acquisition metadata, cancer stage information, quantitative tumor characteristics, and prognostic or therapy-response predictions.

In some embodiments, predictions generated by the artificial intelligence models stored in the resident models 218 may include cancer TNM stage, automatic detection and segmentation of cancer lesions, tumor characteristics including heterogeneity and degree of expression of tumor markers, automatic calculation of cancer prognosis based on imaging findings and clinical history, automatic prediction of response to cancer therapy, and automatic evaluation of efficacy of cancer therapy. For example, the first predictive model may generate an output image comprising a plurality of pixels, each pixel corresponding to tissue with cancer lesions or non-cancerous tissue. The output image may be a segmentation mask where each pixel is assigned a classification label indicating whether the corresponding tissue region contains cancerous cells or represents healthy, non-cancerous tissue. In some embodiments, the first predictive model may assign probability values to each pixel, where the probability value indicates a likelihood that the pixel corresponds to cancerous tissue. The first predictive model may apply a threshold to the probability values to generate binary classifications for each pixel, thereby producing a segmented output image that delineates boundaries between cancerous and non-cancerous regions within the medical image data.

The second predictive model may generate at least one prognostic or therapy-response prediction for the patient based on the quantitative tumor characteristics and the clinical patient data. For example, the second predictive model may analyze the quantitative tumor characteristics extracted from the segmented lesions together with the clinical patient data including patient demographics, cancer history, laboratory test results, and genetic markers to generate predictions about patient outcomes and treatment responses. The second predictive model may generate quantitative metrics indicating the percentage change in total tumor burden between imaging studies, the appearance or disappearance of lesions compared to baseline or prior imaging, changes in standardized uptake values (SUV) for individual lesions, per-lesion longitudinal comparisons (e.g., longitudinal legion associations) that may indicate changes in size, metabolic activity, tumor marker expression, (or other comparisons) for segmented lesions between a current image and prior images provided as input to the model, or composite scores that integrate multiple response indicators into a single therapy-response prediction. The predictions may be incorporated automatically into the medical report generated by the resident device 200.

In some embodiments, the first predictive model and the second predictive model may be implemented as separate, distinct models. For example, the first predictive model and the second predictive model may be executed sequentially within the processing pipeline, where the output of the first predictive model serves as input to the second predictive model. In other embodiments, the first predictive model and the second predictive model may be implemented as a combined model comprising a unified neural network architecture with shared feature extraction layers and task-specific output heads, where a common encoder processes the image data and clinical patient data, and separate decoder branches generate the segmentation output and the prognostic or therapy-response predictions respectively. For example, a multi-task learning architecture may be employed where the combined model simultaneously learns to perform image segmentation and prognostic prediction, leveraging shared representations to improve performance on both tasks while reducing computational overhead compared to executing separate models. In yet other embodiments, the first predictive model and the second predictive model may each comprise an ensemble of multiple models, where the ensemble aggregates predictions from multiple constituent models using techniques such as averaging, voting, or stacking to generate more robust and accurate outputs than any single model alone. For example, the first predictive model may comprise an ensemble of neural networks trained on different subsets of training data or with different architectural configurations, and the segmentation output may be generated by aggregating the pixel-wise predictions from each constituent model in the ensemble.

In some embodiments, the processors (e.g., processors 206) may dynamically select between using separate models, a combined model, or ensemble models based on computational resource availability, latency requirements, or accuracy thresholds specified for a particular clinical application. For example, when the resident device 200 has limited computational resources, the system may execute lightweight separate models sequentially to minimize memory consumption, whereas when the cloud service 300 performs the processing with access to greater computational resources, the system may execute ensemble models to maximize prediction accuracy. The selection between model architectures may also depend on the clinical indication, where initial staging applications requiring high segmentation accuracy may utilize ensemble models for the first predictive model, while treatment response monitoring applications requiring rapid turnaround may utilize a combined model with shared feature extraction to reduce overall processing time.

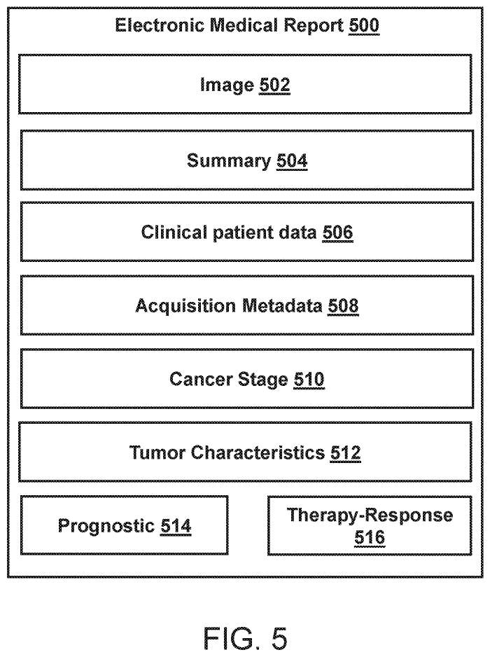

The resident applications 216 may include report components 222 configured to generate electronic reports (e.g., electronic medical report 500 (FIG. 5), medical reports, imaging reports, cancer reports, etc.). The report components 222 may be responsible for generating electronic medical reports based on data processed by the processors 206 and stored in the resident data 220. For example, the report components 222 may retrieve predefined templates from the resident data 220, where the predefined templates define the predetermined structure of the electronic medical report including designated sections for clinical patient data, acquisition metadata, cancer stage information, quantitative tumor characteristics, and prognostic or therapy-response predictions. The report components 222 may parse the predefined templates to identify placeholder fields corresponding to each data category, retrieve the corresponding data elements from the resident data 220, and populate the placeholder fields with the retrieved data elements to generate a complete electronic medical report. The report components 222 may perform data formatting operations to ensure that the populated data conforms to the expected format specifications defined in the predefined templates, including converting numerical values to appropriate units, formatting date and time fields according to standardized conventions, and structuring textual descriptions according to predefined formatting rules. The report components 222 may further validate the completeness of the generated electronic medical report by verifying that all required fields have been populated with valid data before transmitting the report to the output components 204 for display or storage. By executing the report components 222 locally on the resident device 200, the system may reduce network latency associated with transmitting patient data to remote servers for report generation, decrease bandwidth consumption by eliminating the need to transfer large imaging files and clinical datasets over the network 140, and enable report generation to proceed even when network connectivity to cloud-based services is unavailable or degraded, thereby improving system reliability and responsiveness for clinicians who require timely access to comprehensive cancer imaging reports.

In some embodiments, the report components 222 may access a large language model (LLM) to automatically generate portions of the electronic medical report. For example, the report components 222 may store one or more prompts (e.g., a predefined template, predefined instructions, user provided instructions or constraints, etc.) in the resident data 220, where each prompt specifies the desired layout, structure, formatting conventions, and content requirements for the electronic medical report. The report components 222 may retrieve a prompt from the resident data 220 and provide the prompt to the LLM along with the clinical patient data, acquisition metadata, cancer stage information, quantitative tumor characteristics, and prognostic or therapy-response predictions extracted by the processors 206. The LLM may process the prompt and the provided data to generate natural language text for inclusion in the electronic medical report, such as narrative summaries describing imaging findings, clinical impressions synthesizing the quantitative tumor characteristics with the patient's clinical history, or recommendations for follow-up imaging or treatment based on the prognostic predictions. The report components 222 may further leverage the LLM to generate the summary 504 (FIG. 5) section of the electronic report 500 (FIG. 5) by providing a prompt that instructs the LLM to consolidate the key clinical information from multiple report sections into a concise overview suitable for rapid review by cancer care teams. By leveraging the LLM to automatically generate narrative content for the electronic medical report, the system may reduce the time required for clinicians to manually compose report text while ensuring that the generated content maintains consistency with institutional reporting standards and clinical terminology conventions specified in the stored prompts.

By leveraging the architecture of the resident device 200, the system may reduce the time and computational resources required for clinicians to access and interpret cancer imaging results. For example, the resident device 200 may automatically import clinical patient data and digital imaging data, process the image data using the predictive models stored in the resident models 218 to extract quantitative tumor characteristics, generate prognostic or therapy-response predictions, and populate a predefined template to produce a structured electronic medical report. In this way, the system transforms the complex, time-consuming, and error-prone process of accessing multiple sources of patient information stored in different locations into a streamlined workflow, reducing network traffic and latency associated with clinicians accessing disparate data sources during clinical decision-making.

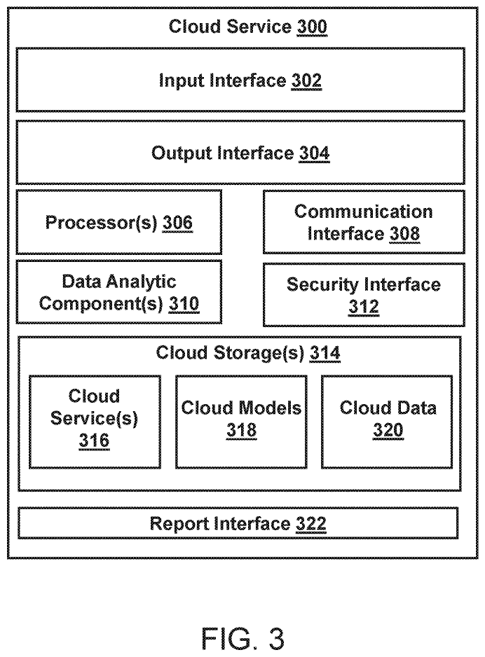

FIG. 3 illustrates a cloud service involved in generating electronic medical reports using positron emission tomography or single-photon emission computed tomography with computed tomography images, in accordance with one or more embodiments. For example, FIG. 3 shows a cloud service 300 involved in generating electronic medical reports using positron emission tomography or single-photon emission computed tomography with computed tomography images. The cloud service 300 includes an input interface 302, an output interface 304, processor(s) 306, a communication interface 308, data analytic component(s) 310, a security interface 312, cloud storage(s) 314, and report interface 322. The cloud storage(s) 314 further includes cloud service(s) 316, cloud models 318, and cloud data 320.

In some embodiments, cloud service 300 may correspond to a cloud-based or server-based service configured to process patient data and generate electronic medical reports. For example, the cloud service 300 may correspond to analytics server 130a described in connection with FIG. 1. The cloud service 300 may be implemented as a server computing device comprising a processor and non-transitory machine-readable storage capable of executing the various tasks and processes described herein. In some embodiments, the cloud service 300 may be any computing device comprising a processor and non-transitory machine-readable storage, such as workstation computers, laptop computers, or server computers. The cloud service 300 may employ various processors such as central processing units (CPU) and graphics processing units (GPU), among others. In some embodiments, the cloud service 300 may operate in a distributed computing environment, such as a cloud environment, where multiple computing devices work together to process data and generate reports.

The input interface 302 may be configured to receive data for processing by the cloud service 300. The input interface 302 may facilitate data ingestion from external sources in cloud-based or server-based environments. For example, the input interface 302 may receive patient data, clinical information, and digital imaging data transmitted from resident devices such as client device 110, imaging device 120a, or clinician workstation 120b via the network 140. The input interface 302 may include application programming interfaces (APIs), web service endpoints, or other data ingestion mechanisms configured to accept data from external systems. In some embodiments where the cloud service 300 is implemented as a computing device, the input interface 302 may include user interfaces for accepting information from users, such as graphical user interfaces (GUIs) that display input fields, dropdown menus, checkboxes, and other interactive elements for data entry.

The input interface 302 may perform the same or similar operations as the input components 202, including the input data interface 202a, the input clinical data interface 202b, and the input imaging data interface 202c described in connection with FIG. 2. For example, cloud service 300 may implement the input interface 302 as a portion of a web application that executes on resident devices such as client device 110, imaging device 120a, or clinician workstation 120b, where the web application provides user interfaces for data entry and facilitates data transmission to the cloud service 300 for centralized processing. In such embodiments, the input interface 302 may receive patient data entered manually by a user such as a radiologist through a web-based graphical user interface rendered on a resident device, perform data validation and format verification operations by comparing extracted input data against predefined format specifications retrieved from the cloud data 320, and store validated data in the cloud data 320 for subsequent processing by the processor(s) 306.

The input interface 302 may import clinical patient data from institution electronic health record (EHR) databases by establishing secure connections via the communication interface 308, retrieving patient demographics, cancer history, laboratory test results, and genetic markers in accordance with a secure application programming interface (API), and performing data format validation and completeness verification before storing the clinical patient data in the cloud data 320. The input interface 302 may also import digital imaging data comprising image data and acquisition metadata by retrieving a plurality of Digital Imaging and Communications in Medicine (DICOM) files from institution Picture Archiving and Communication Systems (PACS), extracting technical information from DICOM metadata including the type of scanner used for image acquisition, the type and amount of radiopharmaceutical injected, and the time of the scanning procedure, and performing a check for DICOM file format compliance before storing the digital imaging data in the cloud data 320 for processing by the processor(s) 306. By implementing the input interface 302 (e.g., as a web application or portion thereof) accessible from resident devices, the cloud service 300 may leverage centralized data validation logic, reduce redundant storage of validation rules across multiple resident devices, and enable consistent data quality enforcement across healthcare facilities for electronic report generation.

The output interface 304 may be configured to provide processed information and generated reports from the cloud service 300. The output interface 304 may facilitate data transmission to external systems in cloud-based or server-based environments. For example, the output interface 304 may transmit electronic medical reports, processed imaging results, and analytical outputs to resident devices, clinician workstations, or other systems via the network 140. In some embodiments where the cloud service 300 is implemented as a computing device, the output interface 304 may include display output components configured to render visual information on screens, monitors, or other display devices. The output interface 304 may perform the same or similar operations as the output components 204 described in connection with FIG. 2. As another example, example, cloud service 300 may implement the output interface 304 as a portion of a web application that executes on resident devices such as client device 110, imaging device 120a, or clinician workstation 120b, where the web application (or portion thereof) provides user interfaces for displaying electronic medical reports and facilitates data transmission from the cloud service 300 to the resident devices. In such embodiments, the output interface 304 may receive processed data from the processor(s) 306 and the cloud data 320, integrate the information in pertinent sections, and automatically generate a standardized medical report for display or transmission. The output interface 304 may render electronic medical reports on a display device associated with a resident device, enabling clinicians to review imaging findings, cancer staging information, and prognostic predictions. By transmitting processed data and generated reports to external devices, the cloud service 300 may leverage centralized processing capabilities while enabling distributed access to report information across healthcare facilities.

The processor(s) 306 may include any hardware or software processors configured to execute operations of the cloud service 300. For example, the processor(s) 306 may include central processing units (CPUs), graphics processing units (GPUs), application-specific integrated circuits (ASICs), field-programmable gate arrays (FPGAs), digital signal processors (DSPs), or any combination thereof. The processor(s) 306 may be configured to process data within the cloud service 300, where data from the input interface 302, the output interface 304, the communication interface 308, the data analytic component(s) 310, the security interface 312, and the cloud storage(s) 314 may be received, transformed, and redirected through the processor(s) 306. The processor(s) 306 may execute applications stored in the cloud service(s) 316 to perform medical report generation functions and other operations. The processor(s) 306 may execute artificial intelligence models retrieved from the cloud models 318 to perform tumor detection, segmentation, classification on medical images, or other operations in accordance with one or more embodiments. In some embodiments, similar to processor(s) 206, the processors 306 may generate one or more metrics for evaluating treatment response in prostate cancer patients based on standardized criteria such as the PCWG3 criteria, the PCWG4 criteria, PPP criteria, or RECIP criteria in conjunction with patient data (e.g., stored in the one or more storages described herein).

The communication interface 308 may facilitate data transmission between the cloud service 300's components, external components, or other devices and systems. For example, the communication interface 308 may establish and manage network connections with other computing devices, servers, databases, and cloud-based services via the network 140. The communication interface 308 may implement various communication protocols and interfaces to enable data exchange, including application programming interfaces (APIs), secure application programming interfaces (secure APIs), representational state transfer (REST) interfaces, and other standardized or proprietary communication protocols. In some embodiments, the communication interface 308 may implement encrypted communication channels using transport layer security (TLS), secure sockets layer (SSL), or other encryption protocols to protect data during transmission. The communication interface 308 may also implement authentication mechanisms, such as token-based authentication, certificate-based authentication, or multi-factor authentication, to verify the identity of external systems before establishing data connections. The communication interface 308 may manage communication sessions with one or more other devices connected to the network 140, including resident devices such as client device 110, imaging device 120a, or clinician workstation 120b, institution databases such as the patient database 150, and public health systems such as the public health server 160. For example, the communication interface 308 may communicate with one or more resident devices to receive patient data, clinical information, and digital imaging data transmitted from healthcare facilities. The communication interface 308 may also communicate with the analytics database 130b to store and retrieve processed data, model outputs, and generated electronic medical reports. In some embodiments, the communication interface 308 may include network interface controllers, wireless communication modules, or other hardware components configured to transmit and receive data packets over wired or wireless network connections via the network 140.

The data analytic component(s) 310 may perform analysis on data received by the cloud service 300. For example, the data analytic component(s) 310 may perform statistical analyses on patient data collected from multiple healthcare facilities at the cloud level. The data analytic component(s) 310 may aggregate and analyze data across institutions to generate insights regarding healthcare practices, imaging procedures, and patient outcomes. Results of analyses performed by the data analytic component(s) 310 may be used to understand at a global level the types of scanning procedures used and how they differ among institutions, to compare results of cancer imaging such as cancer stage distributions among private practice centers versus academic institutions, or to identify trends in patient demographics, treatment patterns, or clinical outcomes across different healthcare settings. The data analytic component(s) 310 may further generate analytical outputs that inform the development of new artificial intelligence models stored in the cloud models 318, where insights derived from the statistical analyses may identify opportunities to improve the care of patients with cancer through enhanced predictive capabilities or refined diagnostic algorithms. In some embodiments, the data analytic component(s) 310 may process medical imaging data, execute queries against the cloud data 320, and generate reports summarizing analytical findings for healthcare administrators, researchers, or public health officials.

The security interface 312 may implement data security measures within the cloud service 300. For example, the security interface 312 may be responsible for data encryption, access control enforcement, and other cyber-defense strategies to guarantee privacy and accuracy of data within the cloud service 300. The security interface 312 may implement encryption protocols for data at rest stored in the cloud storage(s) 314 and data in transit communicated via the communication interface 308. For example, the security interface 312 may encrypt patient clinical data, digital imaging data, and electronic medical reports using cryptographic algorithms such as Advanced Encryption Standard (AES) or other industry-standard encryption methods. The security interface 312 may also implement access control mechanisms that restrict access to patient data based on user roles, permissions, and authentication credentials. For example, the security interface 312 may verify user identities through multi-factor authentication before granting access to sensitive patient information or allowing modifications to electronic medical reports. The security interface 312 may maintain audit logs that record access attempts, data modifications, and system events to enable security monitoring and compliance verification. The security interface 312 may further implement intrusion detection and prevention capabilities to identify and respond to unauthorized access attempts or malicious activities targeting the cloud service 300. By implementing these security measures, the security interface 312 may ensure that patient data processed by the cloud service 300 is protected in accordance with applicable privacy requirements, including healthcare data protection regulations and institutional security policies.

The first predictive model and the second predictive model stored in the cloud models 318 may be the same as, or similar to, the first predictive model and the second predictive model stored in the resident models 218 described in connection with FIG. 2. The processor(s) 306 may execute the cloud models 318 to generate one or more outputs, including segmentation outputs, cancer stage determinations, prognostic predictions, and therapy-response predictions. In some embodiments, the first predictive model and the second predictive model may be implemented as separate, distinct models executed sequentially within the processing pipeline, where the output of the first predictive model serves as input to the second predictive model. In other embodiments, the first predictive model and the second predictive model may be implemented as a combined model comprising a unified neural network architecture with shared feature extraction layers and task-specific output heads. In yet other embodiments, the first predictive model and the second predictive model may each comprise an ensemble of multiple models that aggregates predictions using techniques such as averaging, voting, or stacking.

The cloud storage(s) 314 may contain cloud service(s) 316, cloud models 318, and cloud data 320. The cloud service(s) 316 may store software applications, microservices, and services that execute on the processor(s) 306. For example, the cloud service(s) 316 may include cloud-based implementations of applications similar to the resident applications 216 described in connection with FIG. 2, but deployed in a distributed cloud computing environment. The cloud service(s) 316 may include microservices for report generation, data validation, data transformation, and other processing functions that support the operations of the cloud service 300. The cloud service(s) 316 may also include web-based applications accessible from resident devices, data management utilities, API gateway services, authentication services, and orchestration services that coordinate workflows across multiple processing components. In some embodiments, the cloud service(s) 316 may include services for managing communication between the cloud service 300 and multiple resident devices, enabling centralized coordination of data collection, model deployment, and report distribution across healthcare facilities.

The cloud models 318 may store configured (e.g., pre-trained) artificial intelligence models, machine learning models, predictive models, statistical models, or other models. For example, the cloud models 318 may include a first predictive model configured to process image data and generate an output image representing segmentations of cancer lesions from non-cancerous tissue, and a second predictive model configured to generate prognostic or therapy-response predictions based on quantitative tumor characteristics and clinical patient data.

The cloud models 318 may serve as a centralized repository for artificial intelligence models that can be updated, trained, and deployed to resident devices such as resident device 200. For example, pre-programmed artificial intelligence models deployed at a resident part level may be updated using patient data collected from multiple resident parts via the cloud models 318. New artificial intelligence models may also be developed at the cloud level to develop new strategies to improve the care of patients with cancer. New or updated artificial intelligence models stored in the cloud models 318 may be deployed to resident devices via the communication interface 308. The cloud models 318 may also facilitate federated learning strategies, where model training is distributed across multiple resident devices while aggregating model updates at the cloud level, enabling the system to leverage networking gains of scale and scope while maintaining data privacy at individual healthcare facilities. By centralizing model management in the cloud models 318, the cloud service 300 may ensure consistent model versions across healthcare facilities, enable rapid deployment of model improvements, and support collaborative model development using data from multiple institutions.

The cloud data 320 may store various types of data used by the cloud service 300 in generating electronic medical reports. In some embodiments, cloud data 320 may be a cloud-accessible storage repository, and may correspond to analytics database 130b, patient database 150, or another database, in accordance with one or more embodiments. For example, the cloud data 320 may store clinical patient data imported from record databases across multiple healthcare facilities, including patient demographics, cancer history, laboratory test results, and genetic markers. The cloud data 320 may store digital imaging data comprising image data and acquisition metadata generated when imaging patients, such as Digital Imaging and Communications in Medicine (DICOM) files retrieved from institution Picture Archiving and Communication Systems. The cloud data 320 may store format specification data indicating expected data structures, encoding requirements, and validation rules for data validation operations performed by the input interface 302. The cloud data 320 may store reference data for semantic validation, including valid patient identifiers, acceptable anatomical region codes, and logical relationships between data fields. The cloud data 320 may store predefined templates used for generating electronic medical reports having a predetermined structure. The cloud data 320 may store intermediate processing results generated during image segmentation, quantitative tumor characteristic extraction, and prognostic or therapy-response prediction operations. The cloud data 320 may store generated electronic medical reports populated with clinical patient data, acquisition metadata, cancer stage information, quantitative tumor characteristics, and prognostic or therapy-response predictions. The cloud data 320 may also store aggregated patient data received from multiple resident parts, enabling the data analytic component(s) 310 to perform statistical analyses across institutions and generate insights regarding healthcare practices, imaging procedures, and patient outcomes. The cloud data 320 may operate under stringent rules of privacy and respect to personal data, with security measures (e.g., security protocols) implemented by the security interface 312 to protect patient information stored in the cloud environment.

The cloud service(s) 316 may include report interface 322 configured to generate electronic reports (e.g., electronic medical report 500 (FIG. 5), medical reports, imaging reports, cancer reports, etc.). The report interface 322 may be responsible for generating electronic medical reports based on data processed by the processor(s) 306 and stored in the cloud data 320. For example, the report interface 322 may retrieve predefined templates from the cloud data 320, where the predefined templates define the predetermined structure of the electronic medical report including designated sections for clinical patient data, acquisition metadata, cancer stage information, quantitative tumor characteristics, and prognostic or therapy-response predictions. The report interface 322 may parse the predefined templates to identify placeholder fields corresponding to each data category, retrieve the corresponding data elements from the cloud data 320, and populate the placeholder fields with the retrieved data elements to generate a complete electronic medical report.

The report interface 322 may perform data formatting operations to ensure that the populated data conforms to the expected format specifications defined in the predefined templates, including converting numerical values to appropriate units, formatting date and time fields according to standardized conventions, and structuring textual descriptions according to predefined formatting rules. The report interface 322 may further validate the completeness of the generated electronic medical report by verifying that all required fields have been populated with valid data before transmitting the report to the output interface 304 for display or storage. By executing the report interface 322 on the cloud service 300, the system may leverage centralized processing capabilities, enable consistent report generation across multiple healthcare facilities, and facilitate aggregation of report data for statistical analysis by the data analytic component(s) 310. The report interface 322 may perform the same or similar operations as report component(s) 222 described in connection with FIG. 2, albeit implemented in a cloud-based or server-side environment rather than on a resident device.

In some embodiments, similar to report component(s) 222, report interface 322 may access a large language model (LLM) to automatically generate portions of the electronic medical report. For example, the report interface 322 may retrieve prompts from the cloud data 320 specifying layout, structure, and content requirements for the report. The report interface 322 may provide the prompt to the LLM along with the clinical patient data, acquisition metadata, cancer stage information, quantitative tumor characteristics, and prognostic or therapy-response predictions extracted by the processor(s) 306. The LLM may generate natural language text for inclusion in the electronic medical report, such as narrative summaries describing imaging findings or clinical impressions synthesizing tumor characteristics with patient history. The report interface 322 may leverage the LLM to generate the summary 504 (FIG. 5) section by providing a prompt instructing the LLM to consolidate key clinical information into a concise overview for cancer care teams.

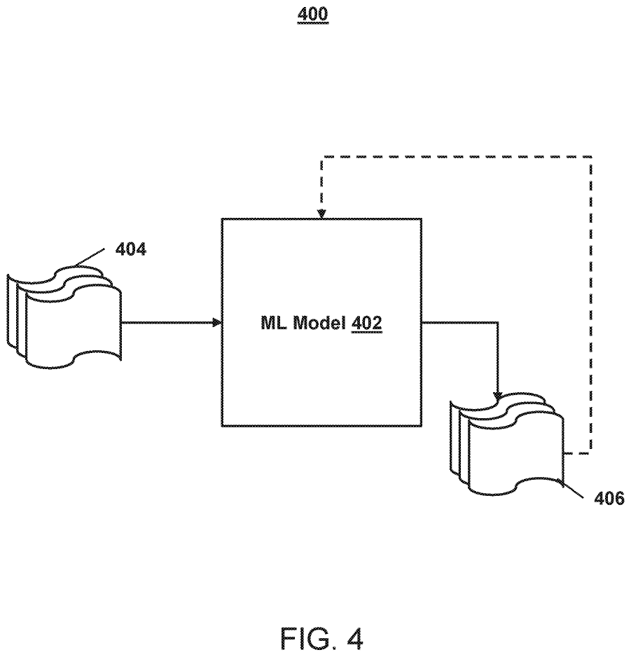

FIG. 4 illustrates an artificial intelligence model that is configured to generate outputs, in accordance with one or more embodiments. Referring to FIG. 4, FIG. 4 shows a model environment 400 showing model 402, which may be a machine learning model, artificial intelligence model, predictive model, etc. (which may be referred collectively as “models” herein). Model 402 may take inputs 404 and provide outputs 406. The inputs may include multiple datasets, such as a training dataset and a test dataset. Each of the plurality of datasets (e.g., inputs 404) may include data subsets related to user data, predicted forecasts and/or errors, and/or actual forecasts and/or errors. In some embodiments, outputs 406 may be fed back to model 402 as input to train model 402 (e.g., alone or in conjunction with user indications of the accuracy of outputs 406, labels associated with the inputs, or with other reference feedback information). For example, the system may receive a first labeled feature input, wherein the first labeled feature input is labeled with a known prediction for the first labeled feature input. The system may then train the first machine learning model to classify the first labeled feature input with the known prediction (e.g., a segmentation of cancer lesions from non-cancerous tissue represented by image data, a cancer stage in accordance with a predefined staging classification, a tumor burden and a degree of expression of at least one tumor marker for segmented lesions, an anatomical region association for each lesion, a prognostic prediction for a patient based on quantitative tumor characteristics and clinical patient data, a therapy-response prediction for a patient, an eligibility state for a patient based on imaging output and prognostic or therapy-response predictions, etc.)

In a variety of embodiments, model 402 may update its configurations (e.g., weights, biases, or other parameters) based on the assessment of its prediction (e.g., outputs 406) and reference feedback information (e.g., user indication of accuracy, reference labels, or other information). In a variety of embodiments, where model 402 is a neural network, connection weights may be adjusted to reconcile differences between the neural network's prediction and reference feedback. In a further use case, one or more neurons (or nodes) of the neural network may require that their respective errors are sent backward through the neural network to facilitate the update process (e.g., backpropagation of error). Updates to the connection weights may, for example, be reflective of the magnitude of error propagated backward after a forward pass has been completed. In this way, for example, the model 402 may be trained to generate better predictions.

In some embodiments, model 402 may include an artificial neural network. In such embodiments, model 402 may include an input layer and one or more hidden layers. Each neural unit of model 402 may be connected with many other neural units of model 402. Such connections can be enforcing or inhibitory in their effect on the activation state of connected neural units. In some embodiments, each individual neural unit may have a summation function that combines the values of all of its inputs. In some embodiments, each connection (or the neural unit itself) may have a threshold function such that the signal must surpass it before it propagates to other neural units. Model 402 may be self-learning and trained, rather than explicitly programmed, and can perform significantly better in certain areas of problem solving, as compared to traditional computer programs. During training, an output layer of model 402 may correspond to a classification of model 402, and an input known to correspond to that classification may be input into an input layer of model 402 during training. During testing, an input without a known classification may be input into the input layer, and a determined classification may be output.

In some embodiments, model 402 may include multiple layers (e.g., where a signal path traverses from front layers to back layers). In some embodiments, back propagation techniques may be utilized by model 402 where forward stimulation is used to reset weights on the “front” neural units. In some embodiments, stimulation and inhibition for model 402 may be more free-flowing, with connections interacting in a more chaotic and complex fashion. During testing, an output layer of model 402 may indicate whether or not a given input corresponds to a classification of model 402 (e.g., a segmentation of cancer lesions from non-cancerous tissue represented by image data, a cancer stage in accordance with a predefined staging classification, a tumor burden and a degree of expression of at least one tumor marker for segmented lesions, an anatomical region association for each lesion, a prognostic prediction for a patient based on quantitative tumor characteristics and clinical patient data, a therapy-response prediction for a patient, an eligibility state for a patient based on imaging output and prognostic or therapy-response predictions, etc.)

In some embodiments, the model (e.g., model 402) may automatically perform actions based on outputs 406. For example, the system may generate an electronic medical report having a predetermined structure in response to obtaining outputs 406 from the model. The outputs 406 may trigger automatic population of predefined report templates with clinical patient data, acquisition metadata, cancer stage information, quantitative tumor characteristics, and prognostic or therapy-response predictions. In some embodiments, the system may automatically store the generated electronic medical report in an electronic data repository for access by authorized clinicians in response to generating outputs 406. The outputs 406 may also trigger automatic transmission of notifications to clinicians indicating that a new report is available for review, or may cause the system to update patient records in the patient database 150 or analytics database 130b with the newly generated predictions and staging information. In some embodiments, the outputs 406 may be used to automatically determine an eligibility state for the patient based on the segmentation output and prognostic or therapy-response predictions, and the system may generate alerts or recommendations regarding treatment eligibility in response to this determination. The outputs 406 may further be used to update training datasets stored in the resident data 220 or cloud data 320 for subsequent model refinement, or may be transmitted to the public health server 160 for aggregation with data from other healthcare facilities. Additionally, the system may automatically generate visualizations, pictorials, or graphical representations of cancer localization based on the segmentation output, thereby providing visualizations in the spatial distribution of the disease (e.g., cancer) across anatomical regions.

In some embodiments, model 402 may be implemented on an ASIC. For example, an ASIC is a specialized hardware chip designed to perform a specific task or set of tasks with high efficiency. Unlike general-purpose processors, such as CPUs or GPUs, ASICs are custom-built for particular applications, optimizing performance, power consumption, and area efficiency. ASICs are widely used in areas such as cryptocurrency mining, telecommunications, and artificial intelligence (AI), where dedicated hardware can provide significant advantages over more flexible but less efficient alternatives. Implementing a large AI model in an ASIC requires designing custom circuits that accelerate the model's computations while ensuring efficient memory management and data movement. Because AI models, particularly deep learning networks, involve extensive matrix multiplications and tensor operations, specialized hardware units such as systolic arrays or tensor processing units (TPUs) can be integrated to optimize these operations. To overcome this challenge, the system may use weight quantization, memory hierarchy optimization, and on-chip interconnects can be employed to improve throughput and reduce power consumption. To accommodate large models, the system may integrate high-bandwidth memory (HBM) or leverage chiplet architectures, where multiple ASICs work together in a modular fashion to process different portions of the model. Training AI models on an ASIC presents significant challenges since training involves dynamic weight updates and high computational flexibility, which contrasts with the fixed nature of ASICs. To do so, the system may use field-programmable gate arrays (FPGAs) or GPUs during the training phase, then transfer the trained model weights to the ASIC for inference. Alternatively, the system may design ASICs that support on-chip fine-tuning or low-bit precision training, allowing for limited retraining directly on the device. Additionally, co-designing hardware and algorithms ensures that the model architecture is tailored to the ASIC's capabilities, reducing inefficiencies and maximizing performance. By integrating specialized training accelerators, approximate computing methods, and efficient dataflow architectures, ASICs can be optimized for both training and inference, enabling large AI models to operate with minimal energy and latency constraints.

As an example with respect to model 402, model 402 may be implemented on an ASIC that is part of a component (e.g., server, or other computing device) of resident device 200, cloud service 300, client device 110, clinician workstation 120b, or analytics server 130a. Each neuron of each of the layers of model 402 may comprise a memory register (e.g., of an electronic storage component) that is configured to store a value associated with clinical data (and/or a bias value), an input portion configured to receive (i) processed values associated with inputs 404 from one or more preceding neurons of a preceding layer or (ii) inputs 404 provided to the model 402, an output portion configured to provide the stored value of the memory register that is associated with the inputs 404 information to (i) one or more subsequent neurons of a subsequent layer or (ii) an output receiving component (e.g., processors 206, processors 306, etc.) to provide the system with output data of model 402. Additionally, each of the connections to the neurons of model 402 (e.g., as indicated by the arrows), may additionally comprise a memory register (e.g., of an electronic storage component) that is configured to store a value associated with processing the input-related information of inputs 404 (e.g., a weight). Each neuron may process the values associated with the model clinical data using at least a portion of a computer processor. For example, where model 402 is implemented on a server that is part of associated with the entity, each neuron may be allocated a portion of processing resources of one or more processors hosted on the server.

During a training routine, model 402 may process clinical data (e.g., medical imaging data, clinical patient data, acquisition metadata, segmentation labels, cancer stage labels, prognostic labels, quantitative tumor characteristics, etc.). For example, clinical data and/or other information is provided as input to model 402, values received by each neuron are processed by respective neurons based on the stored weights of the connections and bias values associated with the neurons. For example, the system may retrieve the weights, biases, or other values from their corresponding memory registers, process the information to generate an updated value (e.g., a value associated with segmenting cancer lesions, classifying cancer stage, or generating prognostic predictions), and provide the updated value to the respective neurons of a subsequent layer during forward propagation. The reverse may be implemented in a similar manner during backpropagation (e.g., to update the weights, biases, or other configurations of model 402). By doing so, the ASIC provides an application-specific platform that is specifically-designed to host model 402 and/or other models involved in processing medical imaging data and generating electronic medical reports, in accordance with one or more embodiments.

In some embodiments, model 402 may be trained. For example, model 402 may be trained during a training routine. The model 402 may be trained on a set of training data. In some embodiments, a first instance of model 402 may be trained to generate an output image, and a second instance of model 402 may be trained to generate prognostic or therapy-response predictions.

In some embodiments, where the first instance of model 402 (e.g., a first predictive model) is trained to generate output images representing segmentations of cancer lesions from non-cancerous tissue represented by image data, the system may use a first set of training data to train the first instance of model 402. For example, the first set of training data may include (i) a set of training medical images (e.g., PET images, CT images, SPECT images, and/or PET/CT images) of patients, (ii) labels indicating target outputs for the training medical images (e.g., segmentations of cancer lesions, non-cancerous tissue segmentations, pixel labels indicating a cancer lesions or non-cancerous tissue, etc.), or other information.

For example, in some embodiments, each training medical image may include a plurality of pixels or voxels, where each pixel or voxel is labeled with (i) a tissue classification indicating whether the pixel corresponds to tissue with cancer lesions or non-cancerous tissue, (ii) an anatomical structure label indicating a type of anatomical structure corresponding to the pixel, and (iii) a lesion label indicating points within the patient associated with metabolic activity indicative of tumor lesions. By using the tissue classification labels in conjunction with the anatomical structure labels and lesion labels, the system may provide contextual information to the model during the training routine, such that the model learns to distinguish between cancer lesions and physiological metabolic activity that may otherwise appear similar in functional images. For example, while certain anatomical structures such as the bladder, kidneys, liver, salivary glands, spleen, and gastrointestinal tract may exhibit normal metabolic uptake in PET scans, the system may use labels indicating anatomical structures to focus segmentation on actual tumor lesions rather than normal physiological activity.

Additionally or alternatively, the system may generate cancer lesion segmentations and cancer stage determinations solely based on the anatomical images (e.g., CT images) and functional images (e.g., PET images or SPECT images) without requiring additional labeled training data indicating anatomical structures. In such embodiments, the first predictive model may be trained to directly process the combined anatomical and functional image data to identify and segment cancer lesions, learning to distinguish between pathological uptake patterns indicative of malignancy and normal physiological uptake patterns based on the spatial, intensity, and morphological characteristics present in the imaging data itself. By doing so, the system may generate more accurate segmentations and cancer stage determinations, as opposed to misclassifying normal metabolic activity as tumor lesions.

The set of training data may be obtained from resident data 220 or cloud data 320 storing model training data. For example, the system may retrieve the set of training data from the cloud data 320 in response to determining the model (e.g., model 402) to be trained. The system may provide the set of training data as input to model 402 during the training routine, and model 402 may generate candidate outputs indicating segmentations of cancer lesions, pixels of the images indicating cancerous tissue, pixels of the images indicating non-cancerous tissue, or other information. During the training routine, model 402 may learn the relationships between the inputs (e.g., image data) and the outputs (e.g., candidate outputs). During the training routine, model 402 may adjust one or more parameters of model 402 to reduce a loss between the candidate outputs and the target outputs (e.g., the labels indicated in the training data) for respective inputs.

In some embodiments, the first predictive model (e.g., the first instance of model 402) may be configured to generate an output image representing segmentations of cancer lesions from non-cancerous tissue represented by the image data. The first predictive model may be configured to generate an output image including a plurality of pixels, where each pixel corresponds to tissue with cancer lesions or non-cancerous tissue. For example, the output image may be a segmentation mask where each pixel is assigned a classification label indicating whether the corresponding tissue region contains cancerous cells or represents healthy, non-cancerous tissue. In some embodiments, the pixels of the output image may be color coded to facilitate ease of clinician review and interpretation. For example, pixels corresponding to cancer lesions may be rendered in a first color (e.g., red) while pixels corresponding to non-cancerous tissue may be rendered in a second color (e.g., green or blue), enabling clinicians to quickly identify and assess tumor locations within the medical image. The color coding of pixels may also reduce error in subsequent processing steps, such as extracting quantitative tumor characteristics, by providing clear visual delineation between cancerous and non-cancerous regions that can be more accurately parsed by downstream processing components. By color coding the segmented pixels, the system may reduce the likelihood of misinterpretation during manual review and improve the accuracy of automated feature extraction operations that rely on the segmentation output.

Training may include iteratively providing data associated with the training medical images to model 402 to cause model 402 to generate outputs, comparing the outputs (e.g., the candidate outputs) with targeted, labeled, outputs representing a ground truth, and updating weights of model 402 to cause model 402 to output updated sets of outputs based on the difference between the outputs and the ground truth that more closely approximate the outputs representing the ground truth. This training may be repeated until model 402 converges (e.g., where the difference between the outputs of model 402 and the ground truth satisfy a difference threshold).

In some embodiments, where the second instance of model 402 (e.g., a second predictive model) is trained to generate at least one prognostic or therapy-response prediction for the patient based on quantitative tumor characteristics and clinical patient data, the system may use a second set of training data to train the second instance of model 402. For example, the second set of training data may include (i) a set of training quantitative tumor characteristics (e.g., tumor burden values, tumor marker expression levels, lesion counts, anatomical region associations, etc.) extracted from medical images of patients, (ii) a set of training clinical patient data (e.g., patient demographics, cancer history, laboratory test results, genetic markers, previous therapy information, etc.), (iii) labels indicating target outputs for the training data (e.g., prognostic outcomes, therapy-response classifications, survival predictions, treatment eligibility determinations, lesion-characteristics, tumor characteristics, etc.), (iv) raw (e.g., unprocessed) anatomical images (e.g., CT images, MRI images) and functional images (e.g., PET images, SPECT images), (v) radiomics features (e.g., texture features, shape features, intensity histogram features, type of radiopharmaceutical, amount of radiopharmaceutical, etc.) or other information.

In some embodiments, where the first predictive model generates the segmentation-masked image (e.g., a multi-label segmentation masked image or color-coded representation), the second predictive model may leverage such labels (e.g., the classification labels corresponding to the pixels in the output images indicating cancerous or non-cancerous tissue) or other encoded (e.g., the color-coded segmentation) image output to more efficiently extract quantitative tumor characteristics for use in generating prognostic or therapy-response predictions. The encoding may reduce computational overhead in the second predictive model by providing pre-classified pixel regions that can be directly parsed without requiring additional segmentation processing, thereby improving the speed and accuracy of downstream prognostic predictions.

During training of the second predictive model, the system may use training data that includes segmentation masked outputs generated by the first predictive model paired with corresponding prognostic outcomes and therapy-response labels. For example, the training data may include multi-label segmentation masked images or color coded-representations thereof, where pixels corresponding to cancer lesions are labeled with labels (e.g., classification labels, color-coded representations, etc.) By training the second predictive model on segmentation masked inputs, the model learns to associate specific color patterns and spatial distributions of colored pixels with prognostic and therapy-response outcomes, enabling the model to bypass redundant feature extraction operations that would otherwise be required to distinguish cancerous from non-cancerous regions, and rather, leverage targeted information pertaining to cancerous over non-cancerous segmentations within image data.

For example, the second predictive model may be trained to recognize that pixels having a first label (e.g., cancerous tissue) represent regions requiring quantitative analysis for tumor burden calculation, while pixels having a second label (e.g., non-cancerous tissue) can be excluded from tumor-related computations, thereby reducing the number of pixels that must be processed during inference. During the training routine, the second predictive model may adjust its parameters to optimize extraction of quantitative tumor characteristics directly from the segmentation masked regions, learning efficient pathways for aggregating tumor burden metrics, calculating tumor marker expression levels, and correlating these characteristics with clinical patient data to generate accurate prognostic or therapy-response predictions. By training the second predictive model to expect and process color-coded inputs from the first predictive model, the system establishes an integrated processing pipeline where the output format of the first predictive model is specifically designed to optimize the input requirements of the second predictive model, reducing overall system latency and computational resource consumption during electronic medical report generation.

For example, in some embodiments, each training sample may include quantitative tumor characteristics paired with clinical patient data, where each training sample is labeled with (i) a prognostic label indicating a predicted patient outcome (e.g., overall survival, progression-free survival, disease recurrence probability, radiographic progression-free survival (rPFS), etc.), (ii) a therapy-response label indicating a predicted response to a specific treatment (e.g., complete response, partial response, stable disease, progressive disease, etc.), and (iii) an eligibility label indicating whether the patient is eligible for a particular cancer therapy based on imaging findings and clinical criteria (e.g., a binary classification, a continuous value, a score, or degree of eligibility based on the imaging findings and/or clinical criteria). By using the prognostic labels in conjunction with the therapy-response labels and eligibility labels, the system may provide contextual information to the model during the training routine, such that the model learns to correlate quantitative tumor characteristics and clinical patient data with patient outcomes and treatment responses. For example, while certain combinations of tumor burden, tumor marker expression, and clinical history may be associated with favorable prognosis, the system may use labels indicating actual patient outcomes to train the model to generate accurate prognostic predictions. By doing so, the system may generate more accurate prognostic and therapy-response predictions, as opposed to relying solely on individual tumor characteristics without considering the interplay between imaging findings and clinical patient data.

In some embodiments, the second predictive model (e.g., the second instance of model 402) may be configured to generate one or more quantitative tumor characteristics based on image data. For example, the second predictive model may be configured to generate one or more quantitative tumor characteristics based on image data, including longitudinal lesion associations (LLAs) that track changes in individual lesions across multiple images captured at different timestamps. For example, the second predictive model may receive as input a set of images comprising a current image ((e.g., CT, MRI, PET/CT, SPECT, SPECT/CT images) and one or more prior images for the same patient captured at earlier timestamps, and generate longitudinal lesion associations that link corresponding lesions between the current image and prior images. The longitudinal lesion associations may indicate what happened to each lesion between two or more timestamps, enabling automated tracking of lesion changes over time. The second predictive model may generate associations between lesions across images captured at different timestamps, derived metrics indicating changes, differences, or percent change, for each lesion (e.g., size, average SUV, lesion diameter, longest axis measurement, lesion disappearance, new lesion appearance), and patient-level conclusions based on aggregated lesion-level changes (e.g., determining that 80% of large lesions disappeared indicates the patient is responding well to therapy). The second predictive model may generate quantitative metrics indicating the percentage change in total tumor burden between images captured at different timestamps, the appearance or disappearance of lesions compared to baseline or prior images, changes in standardized uptake values (SUV) for individual lesions, per-lesion longitudinal comparisons indicating changes in size, metabolic activity, tumor marker expression, lesion diameter, and longest axis measurements for segmented lesions between a current image and prior images provided as input to the model, or composite scores that integrate multiple response indicators into a single therapy-response prediction.

The second set of training data for training the second predictive model to generate longitudinal lesion associations may include (i) sets of training images comprising current and prior images for patients with known lesion correspondences, (ii) labels indicating ground truth lesion associations linking corresponding lesions across timestamps, (iii) labels indicating derived metrics for each lesion including changes in size, SUV, metabolic activity, and tumor marker expression between images captured at different timestamps, and (iv) labels indicating patient-level outcomes based on aggregated lesion-level changes. During the training routine, the second predictive model may learn to identify corresponding lesions across images captured at different timestamps based on spatial location, anatomical region, and lesion characteristics, and to generate accurate derived metrics and patient-level conclusions based on the longitudinal lesion associations. The predictions generated by the second predictive model, including the longitudinal lesion associations, derived metrics, and patient-level conclusions, may be incorporated automatically into the medical report generated by the resident device 200.

The second set of training data may be obtained from resident data 220 or cloud data 320 storing model training data. For example, the system may retrieve the second set of training data from the cloud data 320 in response to determining the second instance of model 402 to be trained. The system may provide the second set of training data as input to the second instance of model 402 during the training routine, and the second instance of model 402 may generate candidate outputs indicating prognostic predictions, therapy-response predictions, eligibility determinations, or other information. During the training routine, the second instance of model 402 may learn the relationships between the inputs (e.g., quantitative tumor characteristics and clinical patient data) and the outputs (e.g., candidate prognostic or therapy-response predictions). During the training routine, the second instance of model 402 may adjust one or more parameters of the second instance of model 402 to reduce a loss between the candidate outputs and the target outputs (e.g., the labels indicated in the training data) for respective inputs.

In some embodiments, the system may receive feedback information to further train model 402. For example, a user may provide, via a user interface, a message to model 402 that includes an accuracy value corresponding to (i) each candidate segmentation of the set of candidate segmentations, (ii) each candidate cancer stage classification of the set of candidate cancer stage classifications, and/or (iii) each candidate prognostic or therapy-response prediction of the set of candidate prognostic or therapy-response predictions that model 402 generated. The accuracy value may be a numerical value, such as a normalized numerical value according to a given scale (0-1, 0-10, 0-100, etc.), a percentage, or other quantitative metric for measuring accuracy. In response to providing the message to the model (e.g., model 402), the system may cause one or more configurations (e.g., weights, biases, or other parameters) of the model to be updated. By doing so, the model may be further trained using quantitative accuracy metrics to better predict or generate segmentations, cancer stage classifications, and prognostic or therapy-response predictions to be used in generating electronic medical reports for cancer imaging.

In some embodiments, the artificial intelligence models (e.g., instances of model 402) may be updated using patient data collected from multiple resident parts via a cloud-based system. For example, pre-programmed artificial intelligence models deployed at a resident part level may be updated using patient data collected from multiple resident parts. In some embodiments, the system may implement federated learning strategies to enable collaborative model training across multiple healthcare facilities while preserving data privacy at each institution. Federated learning is a distributed machine learning approach where model training occurs locally on resident devices using local patient data, and only model parameters or gradients are transmitted to a central server rather than raw patient data. By implementing federated learning, the system may leverage the collective knowledge embedded in patient data across multiple healthcare facilities without requiring the transfer of sensitive patient information to a centralized location, thereby maintaining compliance with healthcare data protection regulations while enabling model improvement.

In some embodiments, the resident models 218 stored on resident device 200 may upload model parameters, such as weights, biases, or gradient updates, to the cloud models 318 stored in cloud storage(s) 314. For example, after a resident device 200 performs local training on patient data stored in resident data 220, the resident device 200 may transmit updated model parameters via communication components 208 to the cloud service 300 for aggregation with parameters received from other resident devices. Alternatively, resident devices may upload their respective training data to the cloud data 320 in a secure and anonymized manner for centralized model training. The cloud service(s) 316 may facilitate this process by implementing secure aggregation protocols that combine model parameters or training data from multiple resident devices without exposing individual contributions. For example, the cloud service(s) 316 may implement cryptographic techniques such as secure multi-party computation or differential privacy mechanisms to aggregate model updates while preventing the reconstruction of individual patient data from the aggregated parameters.

The cloud models 318 may be trained using the aggregated parameters or training data received from multiple resident devices. For example, the processor(s) 306 of cloud service 300 may execute training routines that combine model updates from resident devices to generate an updated global model with improved accuracy and generalizability. The cloud service(s) 316 may coordinate the aggregation process by receiving model parameters from participating resident devices, validating the received parameters, performing weighted averaging or other aggregation operations, and updating the cloud models 318 with the aggregated parameters. Once the cloud models 318 have been updated through the federated learning process, the updated models may be deployed to resident devices via the communication interface 308. For example, the cloud service 300 may transmit updated model weights to resident device 200 via network 140, and the resident device 200 may update the resident models 218 with the received parameters. New artificial intelligence models may also be developed at the cloud level using the aggregated data to develop new strategies to improve the care of patients with cancer. New or updated artificial intelligence models stored in the cloud models 318 may be deployed to resident devices via communication modules. By doing so, the system may leverage data from multiple healthcare facilities to improve the accuracy and generalizability of the predictive models used in generating electronic medical reports for cancer imaging while maintaining data privacy and security at each participating institution.

FIG. 5 illustrates an electronic medical report 500 generated using positron emission tomography or single-photon emission computed tomography with computed tomography images, in accordance with one or more embodiments. As shown in FIG. 5, the electronic medical report 500 includes an image 502, a summary 504, clinical patient data 506, acquisition metadata 508, a cancer stage 510, tumor characteristics 512, a prognostic 514, and a therapy-response 516, or other information.

The image 502 may include one or more medical images associated with the patient. For example, the image 502 may include functional images such as PET images showing metabolic activity, CT images providing anatomical detail, SPECT images depicting physiological processes, or combined PET/CT images that fuse functional and anatomical information. The image 502 may further include segmented images representing segmentations of cancer lesions from non-cancerous tissue, where the segmented images are generated by executing a first predictive model on the image data. The image 502 may include maximum intensity projections that provide a two-dimensional representation of three-dimensional volumetric data, enabling clinicians to visualize the distribution of radiotracer uptake throughout the patient's body. The image 502 may additionally include pictorials providing a graphical representation of cancer localization in the human body, where the pictorials depict anatomical regions affected by cancer lesions in a standardized visual format that facilitates rapid interpretation by cancer care teams. For example, the pictorials may include schematic diagrams of the human body with highlighted regions indicating the anatomical locations of detected tumors, such as primary tumor sites, lymph node involvement, and metastatic lesions. The pictorials may be color-coded to indicate different characteristics of the lesions, such as tumor burden, degree of expression of tumor markers, or response to therapy. In implementations involving prostate cancer imaging, the pictorials may depict the prostate gland, pelvic lymph nodes, bone structures, and other anatomical regions commonly affected by prostate cancer metastases. In implementations involving breast cancer imaging, the pictorials may depict the breast tissue, axillary lymph nodes, chest wall, and common sites of distant metastases such as bone, liver, and lung. In implementations involving lymphoma imaging, the pictorials may depict lymph node stations throughout the body, the spleen, and extranodal sites of disease involvement.

The summary 504 may include a summary of the information in the electronic medical report 500. For example, the summary 504 may include a summary based on the image 502, the clinical patient data 506, the acquisition metadata 508, the cancer stage 510, the tumor characteristics 512, the prognostic 514, the therapy-response 516, or other information. The summary 504 may be generated by one or more models as described herein, such as one or more large language models, summarization models, natural language generation models, or other artificial intelligence models configured to synthesize information from multiple data sources into a consolidated textual description. The summary 504 may provide an overview of imaging findings that consolidates the key clinical information from the report into a concise format for rapid review by cancer care teams.

The clinical patient data 506 may include clinical history providing relevant information about patients and cancer history. For example, the clinical patient data 506 may include patient demographics such as age, gender, weight, height, and comorbidities including diabetes, hypertension, cardiovascular disease, or other conditions that may affect treatment decisions. The clinical patient data 506 may include cancer history such as time of diagnosis, initial diagnosis details, pathology results, cancer stage at diagnosis, and treatment history including prior surgical procedures, chemotherapy regimens, radiation therapy courses, hormonal therapy, immunotherapy, or targeted therapy. In some embodiments involving prostate cancer imaging, the clinical patient data 506 may include cancer history data such as time of diagnosis of prostate cancer and serum PSA value at time of diagnosis, laboratory test results such as serum prostate-specific antigen (PSA) values, previous therapy information including surgery (radical prostatectomy), chemotherapy (e.g., docetaxel, cabazitaxel), and hormonal therapy (e.g., ADT, abiraterone, enzalutamide), and pathology results such as Gleason score at diagnosis or genetic mutation information (e.g., ATM and TP53 mutations).

The clinical patient data 506 may further include clinical state information indicating the current disease status, such as primary prostate cancer, biochemical recurrence, metastatic or nonmetastatic hormone-sensitive prostate cancer, or metastatic or nonmetastatic castration-resistant prostate cancer. In some embodiments involving breast cancer imaging, the clinical patient data 506 may include cancer history such as time of diagnosis of breast cancer, initial mammogram and ultrasound results, laboratory tests, previous therapies including surgery (mastectomy), chemotherapy, hormonal therapy, and immunotherapy, and pathology results such as estrogen receptor (ER) status, progesterone receptor (PR) status, and human epidermal growth factor receptor (HER2) status at diagnosis. In some embodiments involving lymphoma imaging, the clinical patient data 506 may include cancer history such as time of diagnosis of lymphoma, lymphoma type, laboratory tests such as complete blood count, previous therapies including surgery, chemotherapy, radiation therapy, and monoclonal antibodies, and pathology results indicating lymphoma type such as Hodgkin's lymphoma, Burkitt lymphoma, follicular lymphoma, diffuse large B-cell lymphoma, mantle cell lymphoma, marginal zone B-cell lymphoma, peripheral T-cell lymphoma, or B-cell lymphoma. The clinical patient data 506 may also include clinical indication for the scan such as initial staging, re-staging of cancer, treatment response evaluation, or surveillance, as well as genetic markers, genomic information including gene mutations and epigenomic data, and molecular profiling results.

The acquisition metadata 508 may include technical information about the process of image acquisition. For example, the acquisition metadata 508 may include the type of imaging technique such as CT, MRI, PET/CT, SPECT, SPECT/CT, or scintigraphy. The acquisition metadata 508 may include scanner information such as scanner type, model, manufacturer, and location of the scanning procedure. The acquisition metadata 508 may include contrast agent information such as the type of contrast agent, quantity administered, and time of injection relative to the scanning procedure. In some embodiments involving PET/CT imaging, the acquisition metadata 508 may include radiopharmaceutical information such as the type of radiopharmaceutical (e.g., 68Ga-PSMA-11, 18F-DCFPyL, 18F-rhPSMA-7.3 for prostate cancer imaging, or 18F-FDG, 18F-FES for breast cancer and lymphoma imaging), the quantity of radiopharmaceutical injected, and the injection time of radiopharmaceutical (approximately 60 minutes prior to scanning time). The acquisition metadata 508 may further include information regarding whether a diuretic was administered for the imaging procedure, patient positioning information during image acquisition, timing parameters of the scanning procedure, reconstruction algorithms applied to the raw imaging data, and quality control measurements obtained during the imaging session. The acquisition metadata 508 may be extracted automatically from metadata of Digital Imaging and Communications in Medicine (DICOM) files accessed from an institution Picture Archiving and Communication System (PACS) within a Radiology Information System (RIS) environment, eliminating the need for manual review and transcription of technical parameters by radiologists.

The cancer stage 510 may include oncological findings summarizing cancer stage in a standardized way following a predefined staging classification. For example, the cancer stage 510 may include staging information in accordance with the TNM classification system of the American Joint Committee on Cancer (AJCC) or Union for International Cancer Control, which classifies cancer based on the extent of the primary tumor (T), the involvement of regional lymph nodes (N), and the presence of distant metastases (M). The cancer stage 510 may include a TNM stage codeline that summarizes cancer stage according to the TNM classification system, providing a standardized alphanumeric code that can be consistently interpreted across healthcare institutions and clinical decision support systems. In some embodiments involving prostate cancer imaging, the cancer stage 510 may follow the AJCC or PROMISE TNM (e.g., PROMISE v1 (miTNM version 1), PROMISE v2 (miTNM version 2), etc.) classification system. In some embodiments involving breast cancer imaging, the cancer stage 510 may follow the AJCC TNM classification system. In some embodiments involving lymphoma imaging, the cancer stage 510 may follow the TNM classification system or the Lugano classification system, which is specifically designed for staging lymphoma based on the number and location of involved lymph node regions and the presence of extranodal disease. The cancer stage 510 may be determined automatically based on the tumor burden represented by each lesion of the anatomical region, where the system aggregates tumor burden information across all anatomical regions to determine an overall cancer stage. By encoding cancer stage information in a standardized machine-readable format, the cancer stage 510 enables downstream clinical decision support systems to consume staging data without requiring custom parsing logic for each report source.

The tumor characteristics 512 may include quantitative tumor characteristics representing a tumor burden and a degree of expression of at least one tumor marker for every segmented lesion. The tumor characteristics 512 may be extracted from the segmented lesions identified in the output image generated by the first predictive model. For example, the system may extract the tumor characteristics 512 by determining a tumor burden and a degree of expression of at least one tumor marker for every segmented lesion, and associating each lesion with an anatomical region within the image data. The tumor characteristics 512 may include descriptive findings providing detailed description of cancer including anatomical localization, size, shape, and relation to other anatomical structures. The tumor characteristics 512 may include oncological findings that characterize each identified lesion, such as the volume of each lesion, the maximum standardized uptake value (SUVmax) indicating metabolic activity, and the mean standardized uptake value (SUVmean) across the lesion volume. The tumor characteristics 512 may include associations of each lesion with an anatomical region within the image data, enabling clinicians to understand the spatial distribution of disease across different organ systems and anatomical compartments.

In some embodiments involving prostate cancer imaging, the tumor characteristics 512 may include total tumor burden metrics, tumor expression of prostate-specific membrane antigen (PSMA), and heterogeneity measurements indicating variability in tumor marker expression across different lesions. In some embodiments involving breast cancer imaging, the tumor characteristics 512 may include total tumor burden, tumor uptake of fluorodeoxyglucose (FDG) or fluoroestradiol (FES), and receptor expression patterns. In some embodiments involving lymphoma imaging, the tumor characteristics 512 may include total tumor burden, tumor uptake of fluorodeoxyglucose (FDG), and metabolic tumor volume measurements. The tumor characteristics 512 may further include measurements of tumor heterogeneity, lesion count by anatomical region, and comparative metrics indicating changes in tumor characteristics relative to prior imaging studies.

In some embodiments, the tumor characteristics may be generated by the second predictive model. For example, the second predictive model may be configured to generate LLAs to track changes in lesions across one or more images. For example, the second predictive model may receive a set of images as input that include a current image (e.g., CT, MRI, PET/CT, SPECT, SPECT/CT images) and one or more historical images. The second predictive model may process the set of images to generate tumor characteristics such as lesion changes over time related to size, average SUV, lesion diameter, axis measurements, lesion disappearance, lesion appearance, and/or patient-level conclusions.

The prognostic 514 may include at least one prognostic prediction for the patient based on the quantitative tumor characteristics and the clinical patient data. For example, the prognostic 514 may include predictions generated by executing a second predictive model that analyzes the quantitative tumor characteristics together with the clinical patient data to generate predictions about patient prognosis. The prognostic 514 may include automatic calculation of cancer prognosis based on imaging findings and clinical history, such as predictions of overall survival, progression-free survival, disease recurrence probability, time to disease progression, rPFS, therapy eligibility, or other prognostics. The prognostic 514 may include predictions derived from nomograms, which are graphical calculation tools that combine multiple prognostic factors to estimate patient outcomes. In some embodiments involving prostate cancer imaging, the prognostic 514 may include predictions of patient prognosis and response to PSMA-targeted therapy generated using nomograms. The prognostic 514 may further include eligibility status for therapy indicating whether the patient is eligible for a certain cancer therapy based on imaging findings, where a recommendation according to established guidelines is made regarding the patient's eligibility status for therapy. For example, the prognostic 514 may indicate whether the patient meets imaging-based criteria for radioligand therapy, targeted therapy, immunotherapy, or other treatment modalities based on the distribution and characteristics of identified lesions. Such eligibility status may be a binary classification, a continuous value, a score, or degree of eligibility based on the imaging findings and/or clinical criteria for the patient receiving one or more therapies. The prognostic 514 may also include risk stratification information that categorizes patients into low-risk, intermediate-risk, or high-risk groups based on the combination of imaging findings and clinical factors.

The therapy-response 516 may include at least one therapy-response prediction for the patient. For example, the therapy-response 516 may include treatment response evaluation describing changes in tumor noticed on images during or after receiving cancer therapy, such as changes in lesion size, number, metabolic activity, or tumor marker expression compared to baseline or prior imaging studies. The therapy-response 516 may include automatic prediction of response to cancer therapy, where the system generates predictions regarding how the patient is likely to respond to specific treatment regimens based on the quantitative tumor characteristics and clinical patient data. The therapy-response 516 may include automatic evaluation of efficacy of cancer therapy, providing assessments of whether current treatment is achieving desired therapeutic effects based on imaging findings. In some embodiments involving prostate cancer imaging, the therapy-response 516 may include metrics for evaluating treatment response based on the PCWG3 criteria, the PCWG4 criteria, PPP criteria, or RECIP criteria, which provide standardized frameworks for assessing treatment response in metastatic castration-resistant prostate cancer incorporating imaging-based assessments of bone lesions, soft tissue lesions, and prostate-specific antigen (PSA) levels. The therapy-response 516 may include response classifications such as complete response, partial response, stable disease, or progressive disease based on changes in lesion size, number, or metabolic activity between imaging studies. In some embodiments involving breast cancer imaging, the therapy-response 516 may include therapy response evaluation following RECIST (Response Evaluation Criteria in Solid Tumors) and PERCIST (PET Response Criteria in Solid Tumors) criteria. In some embodiments involving lymphoma imaging, the therapy-response 516 may include therapy response evaluation following Lugano criteria and Deauville score, which assess treatment response based on changes in FDG uptake relative to reference tissues. The therapy-response 516 may further include quantitative metrics indicating the percentage change in total tumor burden, the appearance or disappearance of lesions, changes in standardized uptake values (SUV) for individual lesions, or composite scores that integrate multiple response indicators into a single therapy-response assessment.

The electronic medical report 500 may include additional information beyond the components described above. For example, the electronic medical report 500 may include patient identification information such as patient name, date of birth, medical record number, and unique patient identifier. The electronic medical report 500 may also include clinician identification information such as the name and credentials of the interpreting radiologist, the referring physician, and other members of the cancer care team involved in the patient's treatment. The electronic medical report 500 may further include administrative information such as the date and time of report generation, report version number, and institutional identifiers. The electronic medical report 500 may additionally include free-text sections for clinical impressions, recommendations for follow-up imaging or additional diagnostic procedures, and notes regarding incidental findings unrelated to the primary cancer diagnosis.

In some embodiments, the electronic medical report 500 may be generated by populating a predefined template that defines the predetermined structure of the report. The predefined template may include designated sections corresponding to each component of the electronic medical report 500, such as sections for the image 502, the summary 504, the clinical patient data 506, the acquisition metadata 508, the cancer stage 510, the tumor characteristics 512, the prognostic 514, and the therapy-response 516. Each section within the predefined template may be associated with a section identifier that uniquely identifies the section and specifies the type of data to be populated within that section. For example, the system may parse the predefined template to identify placeholder fields within each section, where each placeholder field includes a field identifier indicating the specific data element to be inserted and a data type specification indicating the expected format for the data element. The system may then retrieve the corresponding data elements from the resident data 220 or cloud data 320 based on the field identifiers, validate that the retrieved data conforms to the data type specifications, and populate the placeholder fields with the validated data elements to generate the complete electronic medical report 500.

The template-based approach to report generation provides several technical advantages over unstructured report generation methods. By defining a standardized template structure with consistent section identifiers and field specifications, the system ensures that electronic medical reports generated across different healthcare institutions and for different patients maintain a uniform schema that can be reliably parsed by downstream clinical decision support systems, treatment planning software, and public health data aggregation systems. The use of section identifiers and field identifiers enables automated validation of report completeness, as the system can verify that all required sections and fields have been populated with valid data before finalizing the report. Furthermore, the template-based approach facilitates interoperability by enabling external systems to extract specific data elements from the report using the standardized identifiers, eliminating the need for custom parsing logic that would otherwise be required to interpret reports with varying structures or field naming conventions.

In some embodiments, in response to the resident device 200 or the cloud service 300 detecting that a predefined template has been altered, the system may automatically propagate the template update to resident devices connected via the network 140. For example, the cloud service 300 may monitor template version information stored in the cloud data 320 and, upon detecting a modification to a template structure, transmit the updated template to resident devices via the communication interface 308. The resident device 200 may receive the updated template via the communication components 208 and store the updated template in the resident data 220, replacing or supplementing the previous template version. In response to receiving the updated template, the resident device 200 or the cloud service 300 may automatically regenerate electronic medical reports that were previously generated using the prior template format, populating the new template structure with the clinical patient data, acquisition metadata, cancer stage, quantitative tumor characteristics, and prognostic or therapy-response predictions associated with each patient. By automatically propagating template updates and regenerating reports according to the new template format, the system ensures that all electronic medical reports across healthcare facilities maintain consistency with the current standardized schema, enabling downstream clinical decision support systems to consume report data without requiring reconfiguration to accommodate outdated report structures.

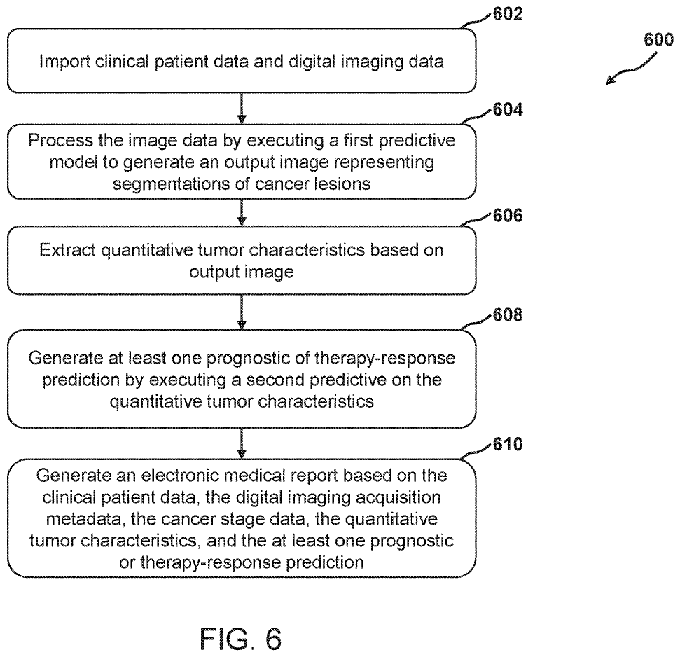

Referring to FIG. 6, illustrated is a flow diagram of a process 600 showing the steps involved in generating electronic medical reports using positron emission tomography or single-photon emission computed tomography with computed tomography images, in accordance with one or more embodiments. The process 600 includes steps (or operations) 602-610. However, other embodiments can include additional or alternative steps or can omit one or more operations altogether. The process 600 is described as being executed by an analytics server, which can be the same as, or similar to, the analytics server 130a described in FIG. 1. However, one or more steps of the process 600 can be executed by any number of computing devices operating in the distributed computing system described in FIG. 1, FIG. 2, FIG. 3, or FIG. 4.

At step 602, process 600 (e.g., using one or more components described above) may import clinical patient data and digital imaging data. For example, the system may import, based on a patient identifier for a patient, clinical patient data from a record database and digital imaging data comprising image data and acquisition metadata generated when imaging the patient. The clinical patient data may be imported automatically from an institution's electronic health record (EHR) system via the input clinical data interface 202b (FIG. 2) or the input interface 302 (FIG. 3). For example, the input clinical data interface 202b may establish a secure connection with the patient database 150 (FIG. 1) via the communication components 208 (FIG. 2) and retrieve clinical patient data associated with the patient identifier. The digital imaging data may include Digital Imaging and Communications in Medicine (DICOM) files accessed from an institution Picture Archiving and Communication System (PACS) within a Radiology Information System (RIS) environment. For example, the input imaging data interface 202c (FIG. 2) may retrieve DICOM files from the patient database 150 (FIG. 1) or the analytics database 130b (FIG. 1) and extract image data and acquisition metadata from the retrieved files. In this way, the system may forgo the ineffective, time-consuming, and error-prone manual review of Electronic Health Records (EHR) and Radiology Information Systems (RIS) that existing systems require radiologists to perform, thereby reducing clinician workload and decreasing the likelihood of transcription errors during report generation.

In some embodiments, the system may retrieve at least patient demographics, cancer history, laboratory-test results, and genetic markers in accordance with a secure application programming interface (API) from the record database. For example, the input clinical data interface 202b (FIG. 2) may invoke a secure API exposed by the patient database 150 (FIG. 1) to retrieve clinical patient data including patient demographics such as age, gender, and comorbidities, cancer history such as time of diagnosis and pathology results, laboratory-test results such as serum biomarker values, and genetic markers such as gene mutations. The communication components 208 (FIG. 2) may implement encrypted communication channels using transport layer security (TLS) to protect the clinical patient data during transmission from the patient database 150 to the resident device 200 (FIG. 2). The import process may be automatic using application programming interface (API) systems implemented by the communication interface 308 (FIG. 3) when the cloud service 300 (FIG. 3) performs the import operation. Data recognition and extraction may be performed using natural language processing and machine learning techniques executed by the processors 206 (FIG. 2) or the processor(s) 306 (FIG. 3), depending on the type of data being extracted. If information required for the medical report cannot be imported automatically, the input clinical data interface 202b may transmit an invocation message to the input data interface 202a (FIG. 2), and the missing information may be introduced manually by a radiologist using the input data interface 202a. The input data interface 202a may perform a check for data format and structure by comparing extracted input data against format specification data stored in the resident data 220 (FIG. 2) before passing the data to the processors 206 and the destination database according to the type of data being entered. In this way, the system may ensure that the clinical patient data is complete and properly formatted for subsequent processing, thereby preventing downstream processing failures and ensuring report accuracy.

In some embodiments, the system may retrieve the digital imaging data. For example, the digital imaging data may be imported by retrieving a plurality of Digital Imaging and Communications in Medicine (DICOM) files associated with the image data and the acquisition metadata. For example, the input imaging data interface 202c (FIG. 2) may establish a connection with the patient database 150 (FIG. 1) via the communication components 208 (FIG. 2) and retrieve DICOM files associated with the patient identifier. The input imaging data interface 202c may parse the DICOM file headers to extract acquisition metadata including the type of scanner used for image acquisition (e.g., PET/CT scanner model and manufacturer), the type and amount of radiopharmaceutical injected (e.g., 68Ga-PSMA-11 or 18F-FDG), the type and amount of contrast agent injected, time of the scanning procedure, and the location of the scanning procedure. The extracted image data and acquisition metadata may be stored in the resident data 220 (FIG. 2) for subsequent processing by the processors 206 (FIG. 2). In this way, the system may automatically extract and provide technical information about the process of image acquisition to the first predictive model, enabling the first predictive model to account for scanner-specific characteristics, radiopharmaceutical uptake timing, and acquisition parameters when generating segmentations of cancer lesions, thereby improving segmentation accuracy and reducing false positive detections that may otherwise result from variations in imaging protocols across different scanners and institutions.

At step 604, process 600 (e.g., using one or more components described above) may process the image data by executing a first predictive model. For example, the processors 206 (FIG. 2) may retrieve a first predictive model from the resident models 218 (FIG. 2) and execute the first predictive model on the image data stored in the resident data 220 (FIG. 2) to generate an output image representing segmentations of cancer lesions from non-cancerous tissue represented by the image data. Alternatively, the processor(s) 306 (FIG. 3) may retrieve the first predictive model from the cloud models 318 (FIG. 3) and execute the model on image data stored in the cloud data 320 (FIG. 3). The first predictive model may correspond to a first instance of the AI model 402 (FIG. 4) that has been trained on training medical images with labels indicating segmentations of cancer lesions and non-cancerous tissue. The first predictive model receives the image data as inputs 404 (FIG. 4) and generates the output image as outputs 406 (FIG. 4). The artificial intelligence models stored in the resident models 218 or cloud models 318 may use as input patient DICOM files accessed from an institution PACS system and processed by the processors 206 or processor(s) 306. In this way, the system may automatically detect and segment cancer lesions on medical images, reducing the time and computational resources required for clinicians to identify tumor locations while providing consistent, reproducible segmentation results across different patients and imaging studies.

In some embodiments, the first predictive model may be configured to generate an output image comprising a plurality of pixels, each pixel corresponding to tissue with cancer lesions or non-cancerous tissue. For example, the first predictive model may perform pixel-wise segmentation where each pixel in the output image is assigned a classification label indicating whether the corresponding tissue region contains cancerous cells or represents healthy, non-cancerous tissue. The output image may be a segmentation mask where pixels corresponding to cancer lesions are rendered in a first color (e.g., red) and pixels corresponding to non-cancerous tissue are rendered in a second color (e.g., green or blue), as described in connection with FIG. 4. The processors 206 (FIG. 2) may store the output image in the resident data 220 (FIG. 2) for subsequent extraction of quantitative tumor characteristics. In this way, the system may provide precise localization of cancer lesions within the image data, enabling accurate calculation of tumor burden and facilitating rapid visual interpretation by clinicians reviewing the segmented images.

At step 606, process 600 (e.g., using one or more components described above) may extract quantitative tumor characteristics. For example, the processors 206 (FIG. 2) may analyze the output image generated by the first predictive model to extract quantitative tumor characteristics representing a cancer stage in accordance with a predefined staging classification based on the output image. The predefined staging classification may include the American Joint Committee on Cancer (AJCC) TNM classification system, which classifies cancer based on the extent of the primary tumor (T), the involvement of regional lymph nodes (N), and the presence of distant metastases (M). The processors 206 may retrieve staging classification rules from the resident data 220 (FIG. 2) and apply the rules to the extracted tumor characteristics to determine the cancer stage. The extracted quantitative tumor characteristics may be stored in the resident data 220 for subsequent use in generating prognostic predictions and populating the electronic medical report. The artificial intelligence models stored in the resident models 218 (FIG. 2) or cloud models 318 (FIG. 3) may perform automatic tumor detection and segmentation on medical images and generate predictions including cancer TNM stage, total tumor burden, and tumor expression of tumor markers. In this way, the system may generate standardized staging information that is less prone to human error compared to manual staging classification, ensuring that accurate and consistent staging information is reliably included in electronic medical reports across different healthcare institutions and practitioners.

In some embodiments, the system may determine a tumor burden and a degree of expression of at least one tumor marker for every segmented lesion. For example, the processors 206 (FIG. 2) may iterate through each segmented lesion identified in the output image and calculate metrics including the volume of the lesion, the maximum standardized uptake value (SUVmax) indicating metabolic activity, and the mean standardized uptake value (SUVmean) across the lesion volume. For prostate cancer imaging, the processors 206 may calculate the degree of expression of prostate-specific membrane antigen (PSMA) for each lesion based on the radiotracer uptake values extracted from the PET image data. The system may also associate each lesion with an anatomical region within the image data by comparing the spatial coordinates of each lesion against an anatomical atlas stored in the resident data 220 (FIG. 2). For example, the processors 206 may determine that a particular lesion is located within the prostate gland, a pelvic lymph node, or a bone structure such as the lumbar spine. The artificial intelligence models stored in the resident models 218 (FIG. 2) may make predictions about cancer anatomical localization, stage, tumor burden, response to cancer therapy, and eligibility for cancer therapy. The extracted tumor characteristics may be stored in the resident data 220 for inclusion in the tumor characteristics 512 (FIG. 5) section of the electronic medical report 500 (FIG. 5). In this way, the system may provide comprehensive quantitative characterization of each tumor lesion for inclusion in the electronic medical report, enabling cancer care teams to assess disease extent and make informed treatment decisions based on objective measurements.

In some embodiments, the system may determine the cancer stage based on the tumor burden represented by each lesion of the anatomical region. For example, the processors 206 (FIG. 2) may aggregate the tumor burden information across all anatomical regions to determine an overall cancer stage in accordance with the predefined staging classification. The processors 206 may sum the tumor volumes across lesions within each anatomical region, count the number of involved lymph node stations, and identify the presence of distant metastases in organs such as the liver, lungs, or bones. Based on this aggregated information, the processors 206 may apply the TNM classification rules stored in the resident data 220 (FIG. 2) to assign T, N, and M stage values and determine an overall stage grouping (e.g., Stage I, Stage II, Stage III, or Stage IV). The determined cancer stage may be stored in the resident data 220 for inclusion in the cancer stage 510 (FIG. 5) section of the electronic medical report 500 (FIG. 5). In this way, the system may generate standardized cancer staging information that can be consistently reported across different patients and healthcare facilities, enabling interoperability with downstream clinical decision support systems that consume staging data for treatment planning.

In some embodiments, the processors 206 may extract longitudinal lesion associations (LLAs) by comparing the current output image against one or more prior output images for the same patient captured at earlier timestamps. For example, the processors 206 may retrieve prior segmentation masks from the resident data 220 (FIG. 2) and perform spatial registration to align the current and prior images to a common coordinate system. The processors 206 may then match corresponding lesions between the current image and prior images based on spatial proximity of lesion centroids, overlap of lesion volumes, and similarity of anatomical region assignments. For each matched lesion pair, the processors 206 may calculate derived metrics indicating changes between timestamps, including percentage change in lesion volume, change in SUVmax, change in SUVmean, change in lesion diameter, and change in longest axis measurement. The processors 206 may further identify new lesions that appear in the current image but have no corresponding lesion in prior images, as well as resolved lesions that were present in prior images but are absent in the current image. The processors 206 may aggregate the lesion-level longitudinal associations to generate patient-level conclusions, such as determining that a threshold percentage of lesions decreased in size indicating positive treatment response, or that new lesions appeared in previously uninvolved anatomical regions indicating disease progression. The extracted longitudinal lesion associations and derived metrics may be stored in the resident data 220 for inclusion in the electronic medical report.

At step 608, process 600 (e.g., using one or more components described above) may generate at least one prognostic or therapy-response prediction. For example, the processors 206 (FIG. 2) may retrieve a second predictive model from the resident models 218 (FIG. 2) and execute the second predictive model to generate at least one prognostic or therapy-response prediction for the patient based on the quantitative tumor characteristics and the clinical patient data. The second predictive model may correspond to a second instance of the AI model 402 (FIG. 4) that has been trained on training data including quantitative tumor characteristics paired with clinical patient data and labels indicating prognostic outcomes and therapy-response classifications. The second predictive model receives the quantitative tumor characteristics and clinical patient data as inputs 404 (FIG. 4) and generates the prognostic or therapy-response prediction as outputs 406 (FIG. 4). For example, the second predictive model may generate predictions of overall survival, progression-free survival, or response classifications such as complete response, partial response, stable disease, or progressive disease based on the Prostate Cancer Working Group 3 (PCWG3) criteria or the Prostate Cancer Working Group 4 (PCWG4) criteria. The artificial intelligence models stored in the resident models 218 or cloud models 318 (FIG. 3) may use predictions obtained from DICOM image analysis together with pre-defined patient clinical data stored in the resident data 220 (FIG. 2) or cloud data 320 (FIG. 3) to make new predictions about patient prognosis and therapy response. The predictions may be stored in the resident data 220 for inclusion in the prognostic 514 (FIG. 5) and therapy-response 516 (FIG. 5) sections of the electronic medical report 500 (FIG. 5), and may be ultimately incorporated automatically in the medical report. In this way, the system may address the technical gap in existing systems that lack the integrated processing architecture necessary to automatically generate prognostic or therapy-response predictions based on quantitative tumor characteristics extracted from imaging data, where the absence of a unified data model connecting image segmentation outputs to clinical data repositories prevented automated correlation of imaging features with patient history for prognostic assessment.

In some embodiments, the system may determine an eligibility state for the patient based on the output image and the at least one prognostic or therapy-response prediction. For example, the processors 206 (FIG. 2) may analyze the output image generated by the first predictive model and the prognostic or therapy-response predictions generated by the second predictive model to determine whether the patient is eligible for a certain cancer therapy based on imaging findings and the prognostic or therapy-response predictions. The processors 206 may retrieve eligibility criteria from the resident data 220 (FIG. 2), where the eligibility criteria specify imaging-based requirements for specific therapies such as radioligand therapy, targeted therapy, or immunotherapy. For example, for prostate cancer patients, the processors 206 may determine eligibility for PSMA-targeted radioligand therapy based on the presence of PSMA-expressing lesions identified in the output image and the predicted therapy response. A recommendation according to established guidelines may be made regarding the patient's eligibility status for therapy, and the eligibility state may be stored in the resident data 220 for inclusion in the electronic medical report. In this way, the system may provide actionable treatment recommendations to cancer care teams based on the integrated analysis of imaging and clinical data, reducing the time required for clinicians to manually evaluate treatment eligibility criteria.

In some embodiments, the second predictive model may generate longitudinal lesion associations (LLAs) by comparing the current output image against one or more prior output images for the same patient captured at earlier timestamps. For example, the second predictive model may receive as input the current output image generated by the first predictive model along with prior segmentation masks retrieved from the resident data 220 (FIG. 2) or cloud data 320 (FIG. 3), and perform spatial registration to align the current and prior images to a common coordinate system. The second predictive model may then match corresponding lesions between the current image and prior images based on spatial proximity of lesion centroids, overlap of lesion volumes, and similarity of anatomical region assignments. For each matched lesion pair, the second predictive model may calculate derived metrics indicating changes between timestamps, including percentage change in lesion volume, change in SUVmax, change in SUVmean, change in lesion diameter, and change in longest axis measurement. The second predictive model may further identify new lesions that appear in the current image but have no corresponding lesion in prior images, as well as resolved lesions that were present in prior images but are absent in the current image. The second predictive model may aggregate the lesion-level longitudinal associations to generate patient-level conclusions, such as determining that a threshold percentage of lesions decreased in size indicating positive treatment response, or that new lesions appeared in previously uninvolved anatomical regions indicating disease progression. The second predictive model may then use the generated longitudinal lesion associations and derived metrics, in conjunction with the quantitative tumor characteristics and the clinical patient data, to generate the at least one prognostic or therapy-response prediction for the patient. For example, the second predictive model may correlate patterns of lesion change over time with clinical outcomes to predict overall survival, progression-free survival, or response classifications such as complete response, partial response, stable disease, or progressive disease. The extracted longitudinal lesion associations, derived metrics, and the at least one prognostic or therapy-response prediction may be stored in the resident data 220 or cloud data 320 (FIG. 3) for inclusion in the electronic medical report.

At step 610, process 600 (e.g., using one or more components described above) may generate an electronic medical report. For example, the report components 222 (FIG. 2) or the report interface 322 (FIG. 3) may generate an electronic medical report having a predetermined structure by populating a predefined template with the clinical patient data, the acquisition metadata, the cancer stage, the quantitative tumor characteristics, and the at least one prognostic or therapy-response prediction. For example, the report components 222 may retrieve a predefined template from the resident data 220 (FIG. 2), where the predefined template defines the predetermined structure of the electronic medical report 500 (FIG. 5) including designated sections for the image 502, the summary 504, the clinical patient data 506, the acquisition metadata 508, the cancer stage 510, the tumor characteristics 512, the prognostic 514, and the therapy-response 516. The report components 222 may parse the predefined template to identify placeholder fields within each section, retrieve the corresponding data elements from the resident data 220, and populate the placeholder fields with the retrieved data elements to generate the complete electronic medical report. The medical report may integrate sections including clinical history providing relevant information about patients and cancer history, technical information about the process of image acquisition, oncological findings summarizing cancer stage in a standardized way following the TNM classification system, descriptive findings providing detailed description of cancer including anatomical localization, treatment response evaluation, eligibility information (e.g., statuses, scores, etc.) for therapy, and patient prognosis. In this way, the system may transform the complex and error-prone process of accessing multiple sources of patient information stored in different locations in a manner that reduces network traffic and latency associated with accessing disparate data sources during clinical decision-making.

In some embodiments, the system may generate the electronic medical report using a large language model (LLM). For example, the report components 222 (FIG. 2) or the report interface 322 (FIG. 3) may retrieve one or more prompts from the resident data 220 (FIG. 2) or the cloud data 320 (FIG. 3), where each prompt specifies the desired layout, structure, formatting conventions, and content requirements for the electronic medical report. The report components 222 or the report interface 322 may provide the prompt to the LLM along with the clinical patient data, the acquisition metadata, the cancer stage, the quantitative tumor characteristics, and the at least one prognostic or therapy-response prediction. The LLM may process the prompt and the provided data to generate natural language text for inclusion in the electronic medical report, such as narrative summaries describing imaging findings, clinical impressions synthesizing the quantitative tumor characteristics with the patient's clinical history, or recommendations for follow-up imaging or treatment based on the prognostic predictions. The report components 222 or the report interface 322 may further leverage the LLM to generate the summary 504 (FIG. 5) section of the electronic report 500 (FIG. 5) by providing a prompt that instructs the LLM to consolidate the key clinical information from multiple report sections into a concise overview suitable for rapid review by cancer care teams.

In some embodiments, the system may store the electronic medical report in an electronic data repository for access by authorized clinicians. For example, the processors 206 (FIG. 2) may transmit the generated electronic medical report via the communication components 208 (FIG. 2) to the cloud data 320 (FIG. 3) or the analytics database 130b (FIG. 1) for centralized storage and access. Alternatively, the processor(s) 306 (FIG. 3) may store the electronic medical report directly in the cloud data 320 (FIG. 3) when the report is generated by the cloud service 300 (FIG. 3). The security components 212 (FIG. 2) or the security interface 312 (FIG. 3) may encrypt the electronic medical report before storage to protect patient data in accordance with applicable privacy requirements. In response to receiving a request for at least a portion of the electronic medical report, the processors 206 or processor(s) 306 may compare a user identifier associated with the request to a predetermined user identifier assigned to the electronic medical report. For example, the security components 212 may retrieve the predetermined user identifier from the resident data 220 (FIG. 2) and compare it against the user identifier provided in the request to verify that the requesting user (e.g., a clinician, patient, etc.) is authorized to access the report. In response to determining the user identifier matches the predetermined user identifier, the system may provide the electronic medical report via the output components 204 (FIG. 2) or the output interface 304 (FIG. 3) to cause a display device associated with the predetermined user identifier to generate a user interface based on the electronic medical report. For example, the output components 204 may render the electronic medical report 500 (FIG. 5) on a display device associated with the client device 110 (FIG. 1) or the clinician workstation 120b (FIG. 1), enabling the authorized clinician to review the imaging findings, cancer staging information, and prognostic predictions. In this way, the system may ensure that patient data is protected in accordance with applicable privacy requirements while enabling authorized clinicians to access the comprehensive medical report through a secure and authenticated access mechanism.

In some embodiments, the electronic medical report may include additional sections based on the prognostic or therapy-response predictions and the eligibility state. For example, the report components 222 (FIG. 2) may generate the electronic medical report based on the clinical patient data, the acquisition metadata, the cancer stage, the quantitative tumor characteristics, the at least one prognostic or therapy-response prediction (e.g., PCWG3, PCWG4, PPP, or RECIP metrics), and an indication of the eligibility state. The report components 222 may populate the prognostic 514 (FIG. 5) section with the prognostic predictions generated by the second predictive model, populate the therapy-response 516 (FIG. 5) section with the therapy-response predictions, and include an eligibility indication within the report structure. The report may include a TNM stage codeline in the cancer stage 510 (FIG. 5) section that summarizes cancer stage according to the TNM classification system, providing a standardized alphanumeric code that can be consistently interpreted across healthcare institutions. The image 502 (FIG. 5) section may include pictorials providing a graphical representation of cancer localization in the human body, where the pictorials depict anatomical regions affected by cancer lesions in a standardized visual format generated based on the lesion-to-anatomical-region associations extracted during step 606. The summary 504 (FIG. 5) section may include a patient summary describing the imaging results, which may be generated by a large language model or natural language generation model executed by the processors 206 (FIG. 2) or processor(s) 306 (FIG. 3). In this way, the system may provide a comprehensive and comprehensible report that consolidates clinical history, technical acquisition information, cancer staging, tumor characteristics, and prognostic predictions into a single document for cancer care teams, enabling efficient clinical decision-making without requiring clinicians to access multiple disparate data sources.

The various illustrative logical blocks, modules, circuits, and algorithm steps described in connection with the embodiments disclosed herein can be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans can implement the described functionality in varying ways for each particular application, but such implementation decisions should not be interpreted as causing a departure from the scope of this disclosure or the claims.

Embodiments implemented in computer software can be implemented in software, firmware, middleware, microcode, hardware description languages, or any combination thereof. A code segment or machine-executable instructions can represent a procedure, a function, a subprogram, a program, a routine, a subroutine, a module, a software package, a class, or any combination of instructions, data structures, or program statements. A code segment can be coupled to another code segment or a hardware circuit by passing and/or receiving information, data, arguments, parameters, or memory contents. Information, arguments, parameters, data, etc., can be passed, forwarded, or transmitted via any suitable means, including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the functions can be stored as one or more instructions or code on a non-transitory computer-readable or processor-readable storage medium. The steps of a method or algorithm disclosed herein can be embodied in a processor-executable software module, which can reside on a computer-readable or processor-readable storage medium. A non-transitory computer-readable or processor-readable media includes both computer storage media and tangible storage media that facilitate the transfer of a computer program from one place to another. A non-transitory processor-readable storage media can be any available media that can be accessed by a computer. By way of example, and not limitation, such non-transitory processor-readable media can comprise RAM, ROM, EEPROM, CD-ROM or other optical disk storage, magnetic disk storage or other magnetic storage devices, or any other tangible storage medium that can be used to store desired program code in the form of instructions or data structures and that can be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm can reside as one or any combination or set of codes and/or instructions on a non-transitory processor-readable medium and/or computer-readable medium, which can be incorporated into a computer program product.

The preceding description of the disclosed embodiments is provided to enable any person skilled in the art to make or use the embodiments described herein and variations thereof. Various modifications to these embodiments will be readily apparent to those skilled in the art, and the principles defined herein can be applied to other embodiments without departing from the spirit or scope of the subject matter disclosed herein. Thus, the present disclosure is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

What is claimed is:

1. A system for generating electronic medical reports using positron emission tomography or single-photon emission computed tomography with computed tomography images, the system comprising:
one or more processors configured to:
import, based on a patient identifier for a patient, clinical patient data from a record database and digital imaging data comprising image data and acquisition metadata generated when imaging the patient;
process the image data by executing a first predictive model to generate an output image representing segmentations of cancer lesions from non-cancerous tissue represented by the image data,
extract quantitative tumor characteristics representing a cancer stage in accordance with a predefined staging classification based on the output image;
generate, by executing a second predictive model, at least one prognostic or therapy-response prediction for the patient based on the quantitative tumor characteristics and the clinical patient data; and
generate an electronic medical report having a predetermined structure by populating a predefined template with the clinical patient data, the acquisition metadata, the cancer stage, the quantitative tumor characteristics and the at least one prognostic or therapy-response prediction.

2. The system of claim 1, wherein the one or more processors are further configured to:
store the electronic medical report in an electronic data repository for access by authorized clinicians;
in response to receiving a request for at least a portion of the electronic medical report, compare a user identifier associated with the request to a predetermined user identifier assigned to the electronic medical report; and
in response to determining the user identifier matches the predetermined user identifier, provide the electronic medical report to cause a display device associated with the predetermined user identifier to generate a user interface based on the electronic medical report.

3. The system of claim 1, wherein the one or more processors configured to import the clinical patient data are configured to:
retrieve at least patient demographics, cancer history, laboratory-test results, and genetic markers in accordance with a secure application programming interface (API) from the record database,
wherein the one or more processors configured to import the digital imaging data are configured to:
retrieve a plurality of Digital Imaging and Communications in Medicine (DICOM) files associated with the image data and the acquisition metadata.

4. The system of claim 1, wherein the first predictive model is configured to generate an output image comprising a plurality of pixels, each pixel corresponding to tissue with cancer lesions or non-cancerous tissue.

5. The system of claim 1, wherein the one or more processors configured to extract the quantitative tumor characteristics are configured to:
for every segmented lesion, determine a tumor burden and a degree of expression of at least one tumor marker; and
associate each lesion with an anatomical region within the image data.

6. The system of claim 5, wherein the one or more processors are further configured to:
determine the cancer stage based on the tumor burden represented by each lesion of the anatomical region.

7. The system of claim 1, wherein the one or more processors are further configured to:
determine an eligibility state for the patient based on the output image and the at least one prognostic or therapy-response prediction,
wherein the one or more processors configured to generate the electronic medical report are configured to:
generate the electronic medical report based on the clinical patient data, the acquisition metadata, the cancer stage, the quantitative tumor characteristics the at least one prognostic or therapy-response prediction, and an indication of the eligibility state.

8. A method for generating electronic medical reports using positron emission tomography or single-photon emission computed tomography with computed tomography images, the method comprising:
importing, by one or more processors and based on a patient identifier for a patient, clinical patient data from a record database and digital imaging data comprising image data and acquisition metadata generated when imaging the patient;
processing, by the one or more processors, the image data by executing a first predictive model to generate an output image representing segmentations of cancer lesions from non-cancerous tissue represented by the image data,
extracting, by the one or more processors, quantitative tumor characteristics representing a cancer stage in accordance with a predefined staging classification based on the output image;
generating, by the one or more processors executing a second predictive model, at least one prognostic or therapy-response prediction for the patient based on the quantitative tumor characteristics and the clinical patient data; and
generating, by the one or more processors, an electronic medical report having a predetermined structure by populating a predefined template with the clinical patient data, the acquisition metadata, the cancer stage, the quantitative tumor characteristics and the at least one prognostic or therapy-response prediction.

9. The method of claim 8, further comprising:
storing, by the one or more processors, the electronic medical report in an electronic data repository for access by authorized clinicians;
in response to receiving a request for at least a portion of the electronic medical report, comparing, by the one or more processors, a user identifier associated with the request to a predetermined user identifier assigned to the electronic medical report; and
in response to determining the user identifier matches the predetermined user identifier, providing, by the one or more processors, the electronic medical report to cause a display device associated with the predetermined user identifier to generate a user interface based on the electronic medical report.

10. The method of claim 8, wherein importing the clinical patient data comprises:
retrieving, by the one or more processors, at least patient demographics, cancer history, laboratory-test results, and genetic markers in accordance with a secure application programming interface (API) from the record database,
wherein importing the digital imaging data comprises:
retrieving, by the one or more processors, a plurality of Digital Imaging and Communications in Medicine (DICOM) files associated with the image data and the acquisition metadata.

11. The method of claim 8, wherein the first predictive model is configured to generate an output image comprising a plurality of pixels, each pixel corresponding to tissue with cancer lesions or non-cancerous tissue.

12. The method of claim 8, wherein extracting the quantitative tumor characteristics comprises:
for every segmented lesion, determining, by the one or more processors, a tumor burden and a degree of expression of at least one tumor marker; and
associating, by the one or more processors, each lesion with an anatomical region within the image data.

13. The method of claim 12, further comprising:
determining, by the one or more processors, the cancer stage based on the tumor burden represented by each lesion of the anatomical region.

14. The method of claim 8, further comprising:
determining, by the one or more processors, an eligibility state for the patient based on the output image and the at least one prognostic or therapy-response prediction,
wherein generating the electronic medical report comprises:
generating, by the one or more processors, the electronic medical report based on the clinical patient data, the acquisition metadata, the cancer stage, the quantitative tumor characteristics the at least one prognostic or therapy-response prediction, and an indication of the eligibility state.

15. One or more non-transitory computer-readable mediums storing instructions thereon that, when executed by one or more processors, cause the one or more processors to:
import, based on a patient identifier for a patient, clinical patient data from a record database and digital imaging data comprising image data and acquisition metadata generated when imaging the patient;
process the image data by executing a first predictive model to generate an output image representing segmentations of cancer lesions from non-cancerous tissue represented by the image data,
extract quantitative tumor characteristics representing a cancer stage in accordance with a predefined staging classification based on the output image;
generate, by executing a second predictive model, at least one prognostic or therapy-response prediction for the patient based on the quantitative tumor characteristics and the clinical patient data; and
generate an electronic medical report having a predetermined structure by populating a predefined template with the clinical patient data, the acquisition metadata, the cancer stage, the quantitative tumor characteristics and the at least one prognostic or therapy-response prediction.

16. The one or more non-transitory computer-readable mediums of claim 15, wherein the instructions further cause the one or more processors to:
store the electronic medical report in an electronic data repository for access by authorized clinicians;
in response to receiving a request for at least a portion of the electronic medical report, compare a user identifier associated with the request to a predetermined user identifier assigned to the electronic medical report; and
in response to determining the user identifier matches the predetermined user identifier, provide the electronic medical report to cause a display device associated with the predetermined user identifier to generate a user interface based on the electronic medical report.

17. The one or more non-transitory computer-readable mediums of claim 15, wherein the instructions that cause the one or more processors to import the clinical patient data cause the one or more processors to:
retrieve at least patient demographics, cancer history, laboratory-test results, and genetic markers in accordance with a secure application programming interface (API) from the record database,
wherein the instructions that cause the one or more processors to import the digital imaging data cause the one or more processors to:
retrieve a plurality of Digital Imaging and Communications in Medicine (DICOM) files associated with the image data and the acquisition metadata.

18. The one or more non-transitory computer-readable mediums of claim 15, wherein the first predictive model is configured to generate an output image comprising a plurality of pixels, each pixel corresponding to tissue with cancer lesions or non-cancerous tissue.

19. The one or more non-transitory computer-readable mediums of claim 15, wherein the instructions that cause the one or more processors to extract the quantitative tumor characteristics cause the one or more processors to:
for every segmented lesion, determine a tumor burden and a degree of expression of at least one tumor marker; and
associate each lesion with an anatomical region within the image data.

20. The one or more non-transitory computer-readable mediums of claim 19, wherein the instructions further cause the one or more processors to:
determine the cancer stage based on the tumor burden represented by each lesion of the anatomical region.
